(19) United States
(12) Patent Application Publication
(10) Pub. No.: US 2005/0009036 A1
(43) Pub. Date: Jan. 13, 2005

(54) Methods and kits for obtaining nucleic acid from biological samples

(75) Inventors: Luz Montesclaros, Pittsburg, CA (US); I. Lawrence Greenfield, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/618,493

(22) Filed: Jul. 11, 2003

(51) Int. Cl. C12Q 1/68; C12N 1/08

(52) U.S. Cl. 435/6; 435/270

(57) ABSTRACT

Methods and kits for isolating nucleic acids from a sample, typically a biological sample are disclosed. In certain embodiments, the methods and kits of the invention comprise at least one protease and at least one solid phase. In certain embodiments, the methods and kits of the invention comprise at least one chaotrope and at least one solid phase. In certain embodiments, the inventive methods and kits further comprise at least one chaotrope, at least one zwitterionic compound, at least one cationic detergent, at least one non-ionic detergent, or combinations thereof.

### DESCRIPTION OF THE INVENTION

Field of the Invention

The present invention generally relates to obtaining, binding, and isolating of nucleic acid sequences from biological samples, including but not limited to clinical, forensic, and research samples. The invention provides methods, reagents, and kits for binding and for isolating nucleic acid sequences in biological samples.

### BACKGROUND OF THE INVENTION

The accuracy and reproducibility of many current molecular biology analytical techniques, such as genotyping and various genomic analyses, pathogen detection and monitoring, and forensic identification, are frequently dependent, at least in part, on the purity, quantity, and quality of the nucleic acid being analyzed. This nucleic acid generally originates from biological or clinical samples, although cultured cells may also serve as the source of nucleic acid.

A variety of nucleic acid isolation techniques have been developed to isolate cellular nucleic acid, frequently genomic DNA (gDNA), from such starting materials. These isolation methods are typically tedious, may require the use of caustic reagents, and may be labor intensive.

Alternative methods for isolating nucleic acids from complex biological samples such as blood and serum have been developed and typically include lysing the cells and inactivating nucleases using protease, chaotropic salts, such as guanidine hydrochloride or guanidinium thiocyanate, and a nonionic surfactant (Boom, R et. Al. 1990, J. Clinical Microbio.p. 495-503). Although these methods eliminate the need for organic solvents, e.g. phenol and/or chloroform, they commonly do not produce the quality and yield of high integrity nucleic acid typically needed for many current molecular biology applications.

Detergents are generally used in cell lysis protocols, as they often disrupt cell membranes and solubilize proteins. The most common detergents used in DNA purification are the ionic detergents, such as sodium dodecyl sulfate (SDS), and the non-ionic detergents such as Triton-X and the Tween series. Anionic detergents are typically effective in solubilizing protein but may precipitate in high salt solutions that are often used to enhance the binding of nucleic acid to various matrices and supports. Most nonionic detergents, such as Triton X, Tween 20 and NP-40, are less effective than the ionic detergents at disrupting protein aggregates. Zwitterionic compounds on the other hand, commonly possess some properties of ionic detergents and some properties of non-ionic detergents.

### SUMMARY OF THE INVENTION

The present invention is directed to methods, reagents, and kits for obtaining and binding and for isolating nucleic acid from samples, including but not limited to, biological fluids, cells, tissues, swabs comprising biological fluids and/or cells, cell culture material, and the like. Typically, nucleic acid is released from the starting material, preferentially reversibly bound to at least one solid phase, and isolated, generally by eluting the nucleic acid from the solid phase.

In certain embodiments of the invention, methods are provided for obtaining nucleic acid from a biological sample and binding the nucleic acid to a solid phase. These methods include combining the sample with at least one protease and at least one zwitterionic compound to form a combination. In certain embodiments, a protease solution comprising at least one protease and at least one zwitterionic compound is combined with the sample. In certain embodiments, the at least one protease and the at least one zwitterionic compound are separately combined with the biological sample, although the separate combining may (but need not) occur simultaneously or near simultaneously. In certain embodiments, the protease solution further comprises at least one first chaotrope. In certain embodiments, at least one first chaotrope is added separately to the combination. The combination may be incubated, if necessary, under conditions appropriate for proteolyzing at least one protein in the combination. Next, the combination is exposed to at least one solid phase and at least one nucleic acid-solid phase complex (“Complex”; plural and collectively, “Complexes”) forms. In certain embodiments, the bound Complexes are separated from the remaining, unbound components of the combination. In certain embodiments, at least one nucleic acid is isolated from at least one Complex.

In certain embodiments of the invention, methods are provided for isolating nucleic acid from a sample comprising combining the sample with at least one first chaotrope and at least one zwitterionic compound to form a combination. The combination is then exposed to at least one solid phase and at least one Complex forms. In certain embodiments, the Complexes are separated from unbound components of the combination. In certain embodiments, at least one nucleic acid is isolated from at least one bound Complex.

In certain embodiments, the combination further comprises at least one cationic detergent. In certain embodiments, the combination further comprises at least one second chaotrope that may be the same or different from the first chaotrope. In certain embodiments, the combination further comprises at least one DNase inhibitor.

The invention also provides kits designed to expedite performing the methods of the invention. Kits serve to expedite the performance of the inventive methods by assembling two or more components required for carrying out the methods. Kits preferably contain components in pre-measured unit amounts to minimize the need for measurements by end-users. Kits preferably include instructions for performing one or more methods of the invention. Preferably, the kit components are optimized to operate in conjunction with one another.

In certain embodiments, kits are provided for obtaining and binding nucleic acids or for isolating nucleic acids according to the methods of the invention. In certain embodiments, kits comprise: at least one zwitterionic compound; at least one first chaotrope, at least one second chaotrope, or at least one first chaotrope and at least one second chaotrope; and at least one solid phase. In certain embodiments, such kits may further comprise at least one protease; at least one cationic detergent; at least one wash solution and/or reagents to make at least one wash solution, including but not limited to at least one alcohol; at least one elution solution and/or reagents to make at least one elution solution, including but not limited to, at least one alkaline buffer, at least one alkaline solution or both at least one alkaline buffer and at least one alkaline solution; or combinations thereof. In certain embodiments the at least one elution solution comprises a low ionic strength buffer.

### BRIEF DESCRIPTION OF THE DRAWINGS

FIG. 1: depicts a schematic overview of exemplary embodiments of the inventive methods. In FIG. 1A, protease solution, comprising at least one protease and optionally at least one zwitterionic compound and/or at least one first chaotrope, is added to a sample to form a combination. A chaotrope solution comprising at least one second chaotrope is added in this illustrative embodiment. When the combination is added to at least one solid phase, at least one Complex forms when at least one nucleic acid binds to the solid phase. Optionally, the remaining unbound materials in the combination are separated and the Complexes are washed. Optionally, at least one nucleic acid is eluted from at least one Complex and isolated. FIG. 1B illustrates another exemplary embodiment, wherein a combination forms when a chaotropic solution and a sample are combined. When the combination is added to at least one solid phase, at least one Complex forms. Optionally, the remaining unbound materials in the combination are separated and the Complexes are washed. Optionally, at least one nucleic acid is eluted from at least one Complex and isolated.

FIG. 2: graphically depicts the average recovery of calf thymus genomic DNA using chaotrope GuSCN and various surfactants in one illustrative embodiment of the invention, described in Example 1. The Y-axis indicates the average recovery of gDNA in micrograms and the X-axis identified the various surfactants employed. A no detergent preparation was included as a control. The bars represent the average amount of gDNA recovered. The error bars represent the 95% confidence interval of the mean of three replicate samples.

FIG. 3: depicts the effects of various zwitterionic compounds on the quantity and quality of gDNA isolated according to one exemplary embodiment, described in Example 3. FIG. 3A graphically depicts the quantity and purity of gDNA isolated from whole blood samples using different zwitterionic detergents. The X-axis identifies the detergents that were used and the Y-axis is the number of μg of gDNA isolated per sample. The number above each sample bar represents the amount of gDNA isolated using the identified zwitterionic detergent and the error bars represent the 95% confidence interval of the mean of three replicates. The purity of the isolated nucleic acid is determined, based on the A260/A280 absorbance ratio (as measured using a spectrophotometer) for each isolated nucleic acid, shown in diamond symbols (♦). FIG. 3B shows a picture of an electrophoretic gel profile obtained when 10 μL of isolated nucleic acid was electrophoresed on a 1% agarose gel, then stained with ethidium bromide. Lane 1 (L to R) contains 1 microgram (μg) of a DNA standard (human gDNA, BD Biosciences Clontech Cat. No. 6550-1); lanes 2 and 15 contain a DNA ladder (300 ng 1 Kb DNA Extension Ladder, Gibco BRL, Cat. No. 10511-012); nucleic acid isolated from duplicate combinations comprising the zwitterionic compounds Empigen BB (lanes 3 and 4), LDAO (lanes 5 and 6), DDMAB (lanes 7 and 8), Zwittergent 3-12 (lanes 9 and 10), Zwittergent 3-14 (lanes 11 and 12), or Zwittergent 3-16 (lanes 13 and 14) are also shown. Unless otherwise noted, all gel electrophoresis described herein was performed using 1% agarose gels that were electrophoresed and stained with ethidium bromide according to conventional methods known in the art.

FIG. 4: graphically depicts the yields in micrograms of nucleic acid isolated from 200 μL fresh blood samples using one exemplary method of the invention, as described in Example 5. Samples were treated in parallel except for the amount of zwitterionic compound in each combination, which varied from 0% to 4.0% (weight/volume), as indicated. The X-axis represents the amount of Zwittergent 3-16 used, in weight/volume percentage. The Y-axis represents the quantity of gDNA recovered in micrograms. Each bar is actually a set of stacked bars, with a white bar (in back) overlaid with a striped bar. The striped bars represent the quantity of gDNA recovered using 1×TE buffer as the elution solution while the white bars represents the quantity of gDNA recovered from the solid phase when subsequently eluted with a very strong base. The two overlying bars in each pair demonstrate that gDNA elution in low ionic strength buffer (1×TE) buffer is very efficient as the height of the striped bar (TE eluate) and corresponding white bar (pooled eluate) are very similar. The error bars represent the 95% confidence interval of the mean of three replicates for the TE eluate.

FIG. 5: depicts the quantity and quality of gDNA isolated from cultured Raji cells according to one exemplary embodiment, described in Example 6. FIG. 5A graphically depicts the quantity of gDNA isolated from decreasing numbers of Raji cells (L to R). The X-axis identifies the number of cells in the sample before extraction, ranging from one million cells to one cell. The Y-axis represents the amount of gDNA recovered in nanograms. Each datapoint represents the average of three separate measurements (i.e., N=3) and the error bars indicate the 95% confidence interval of the mean of the three separate samples.

FIG. 5B shows a picture of an electrophoretic gel profile obtained when 10 μL of isolated nucleic acid was electrophoresed on a 1% agarose gel, then stained with ethidium bromide. From left to right the lanes contain: (lane 1) 1 μg human gDNA (control); (lane 2) molecular weight ladder (300 ng 1 Kb DNA Extension Ladder, Gibco BRL, Cat. No. 10511-012); and 10 μL aliquots of eluate obtained from triplicate samples comprising: 1,000,000 Raji cells (lanes 3-5); 500,000 Raji cells (lanes 6-8); 100,000 Raji cells (lanes 9-11); and 50,000 Raji cells (lanes 12-15).

FIG. 6: graphically depicts the quality and yield of isolated nucleic acid from whole blood samples using varying quantities of silica particles according to one exemplary embodiment, described in Example 7. The X-axis indicates the volume of blood sample and the Y-axis indicates the quantity of isolated gDNA in μg. The diamond symbol (♦) indicates results obtained using 5 mg silica particles, the square symbol (□) indicates results obtained using 7 mg silica particles, and the triangle symbol (Δ) indicates the results obtained using 10 mg of silica particles.

FIG. 7: graphically depicts the quality and quantity of nucleic acid isolated from previously frozen whole blood containing one of three different anticoagulants, citrate, EDTA, or heparin, with and without protease digestion, as described in Example 8. The X-axis indicates the presence (“(+) PK”) or absence (“(−) PK”) of protease in the combination and also the identity of the anticoagulent in the blood sample. The left Y-axis represents the quantity of nucleic acid isolated, measured in μg and correspond to the height of the bars for each condition tested, as shown on the X-axis. The right Y-axis indicates the A260/A280 absorbance ratio and corresponds to the diamond symbols (♦) above each condition tested, as shown on the X-axis. The bars represent the average yield of three replicate samples for each condition tested and the associated error bars represents the 95% confidence interval for those replicates.

FIG. 8: graphically depicts the quantity and quality of nucleic acid isolated from whole blood using increasing concentrations of chaotrope according to one exemplary embodiment, described in Example 9. The X-axis represents the final molar concentration of chaotrope (GuSCN in this example). The left Y-axis represents the quantity of nucleic acid isolated, measured in μg and correspond to the height of the bars for each chaotrope concentration, shown on the X-axis. The right Y-axis indicates the A260/A280 absorbance ratio and corresponds to the diamond symbols (♦) above each chaotrope concentration shown on the X-axis. Each diamond symbol represents the average of three replicates. The error bars represent the 95% confidence interval of the means of three replicates.

FIG. 9: graphically depicts the quantity of nucleic acid isolated from whole blood samples using chaotrope solutions with differing pH, as described in Example 10. The X-axis indicates the pH of the chaotrope solution buffer, ranging from pH 6.0 to pH 10.0. The Y-axis represents the quantity of nucleic acid isolated, in μg. The error bars represent the 95% confidence interval of the means of three replicates.

FIG. 10: depicts the quantity and quality of gDNA isolated from fresh and previously frozen whole blood samples collected in the presence of an anticoagulant (citrate phosphate dextrose (CPD), EDTA, or heparin) according to one exemplary embodiment, described in Example 11. FIG. 10A graphically depicts the quantity of gDNA isolated from the fresh and frozen blood samples. The X-axis identifies the quantity of gDNA recovered, in μg. The left panel on the Y-axis corresponds to fresh whole blood samples collected in CPD, EDTA, or Heparin; and the right panel on the Y-axis corresponds to frozen whole blood samples collected in CPD, EDTA, or Heparin. The bar shading represents the donor of the blood sample, as indicated. The height of each bar represents the average of thirty-two separate nucleic acid isolation procedures. The error bars represent the 95% confidence interval of the means.

FIG. 10B shows a picture of three electrophoretic gel profiles, a, b, and c, that correspond to frozen whole blood samples collected in Citrate, EDTA, or Heparin, respectively. Ten μL of eluate comprising nucleic acid isolated from appropriate samples (or markers) were electrophoresed on a 1% agarose gel, then stained with ethidium bromide. From left to right the lanes contain: (lanes 1 and 20) molecular weight markers (300 ng 1 Kb DNA Extension Ladder, Gibco BRL, Cat. No. 10511-012); (lanes 2-19) eluates from separate isolations from appropriate samples, as indicated.

FIG. 11: depicts the yield, purity, and quality of gDNA isolated from whole blood samples obtained from different species, including mouse, rat, rabbit, chicken, dog, pig, sheep, horse, cow, and human, as described in Example 12. FIG. 11A graphically depicts the quantity and purity of the isolated nucleic acid. The X-axis indicates the species from which the blood sample was obtained. The left Y-axis indicates the quantity of nucleic acid recovered, in micrograms. The right Y-axis indicates the purity of the isolate nucleic acid, as determined by the A260/A280 absorbance ratio. The height of each bar corresponds to the amount of nucleic acid recovered from the indicated blood sample. The diamond symbol (♦) above each bar indicates the A260/A280 ratio for the corresponding eluate.

FIG. 11B depicts the electropherotic profile of the eluates being tested. Lanes 1-3 (L to R) each contain 1 μg of a standard for pig, cow, or human gDNA (Clontech Cat No. 6651-1; Clontech Cat No. 6850-1; BD Biosciences Clontech Cat. No. 6550-1), respectively; lanes 4 and 15 contain a DNA ladder (300 ng 1 Kb DNA Extension Ladder, Gibco BRL, Cat. No. 10511-012); lanes 5-14 contain a 20 μL aliquot of the nucleic acid isolated from mouse, rat, rabbit, chicken, dog, pig, sheep, horse, cow, and human blood samples, respectively. The skilled artisan will appreciate that chicken red blood cells are nucleated, in contrast to the other species tested. Thus, a given volume of chicken blood will contain much more gDNA than the same volume of blood from a mouse, rat, rabbit, etc.

FIG. 12: depicts the yield, purity, and quality of gDNA isolated from HeLa, PC3, or Raji cultured human cells lines, as described in Example 13. FIG. 12A graphically depicts, in a log plot format, the quantity and purity of nucleic acid isolated from the three cell types according to this exemplary embodiment. The X-axis indicates the number of cells in the sample from which the corresponding nucleic acid was obtained, from 10 cells to one million (1000000) cells. The Y-axis indicates nanograms (ng) of nucleic acid recovered. Results from HeLa cells are indicated by the diamond symbol (♦); PC3 cells are indicated by the square symbol (□); and Raji cells are indicated by the triangle symbol (Δ).

FIG. 12B is a picture of three electropherotic gel profiles, annotated: a. HeLa cells; b. PC3 cells; and c. Raji cells. Each gel was loaded with isolated nucleic acid obtained from the indicated cell types. Lane 1 (L to R) of each of the gels contains 1 microgram (μg) of a DNA standard (human gDNA, BD Biosciences Clontech Cat. No. 6550-1); lanes 2 and 15 of each gel contain a DNA ladder (300 ng 1 Kb DNA Extension Ladder, Gibco BRL, Cat. No. 10511-012); lanes 3-8 contain eluates isolated from separate combinations comprising 10⁶ cells of the corresponding cell line; and lanes 9-14 contain eluates isolated from separate combinations comprising 10⁵ cells of the corresponding cell line.

FIG. 13: depicts the yield, purity and quality of nucleic acid obtained from whole blood samples using various solid phase materials. FIG. 13A graphically depicts the quantity and purity of gDNA isolated from whole blood samples using different silica-based solid phase materials. The X-axis identifies the solid phase materials, the left Y-axis indicates the yield of gDNA in μg, and right Y-axis indicates the 260/280 absorbance ratios. The error bars represent the 95% confidence interval of the mean of three replicates. The diamond symbols (♦) indicate the 260/280 absorbance ratio for the isolated nucleic acid, as measured using a spectrophotometer.

FIG. 13B shows a picture of an electrophoretic gel profile obtained when 10 μL of isolated nucleic acid was electrophoresed on a 1% agarose gel. Lane 1 (L to R) contains 1 μg of a DNA standard (human gDNA, Clontech Cat. No. 6550-1, BD Biosciences Clontech, Palo Alto, Calif.); lane 2 contains a DNA ladder (300 ng 1 Kb DNA Extension Ladder, Gibco BRL, Cat. No. 10511-012); lanes 3-17 contain nucleic acid that was isolated from triplicate combinations Sigma silica, Cerac silica S-2046, diatomaceous earth, Celite Grade SW-10 and GF/B membrane.

### DETAILED DESCRIPTION OF THE INVENTION

The section headings used herein are for organizational purposes only and are not to be construed as limiting the subject matter described. All literature and similar materials cited in this application, including but not limited to, patents, patent applications, articles, books, and treatises, regardless of the format of such literature and similar materials, are expressly incorporated by reference in their entirety for any purpose.

Definitions

The term “biological sample” is used in a broad sense and is intended to include a variety of biological sources that contain nucleic acids. Exemplary biological samples include, but are not limited to, whole blood; red blood cells; white blood cells; buffy coat; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, and the like. The skilled artisan will appreciate that lysates, extracts, or material obtained from any of the above exemplary biological samples are also within the scope of the invention. Tissue culture cells, including explanted material, primary cells, secondary cell lines, and the like, as well as lysates, extracts, or materials obtained from any cells, are also within the meaning of the term biological sample as used herein. Microorganisms and viruses that may be present on or in a biological sample are also within the scope of the invention. Materials obtained from clinical or forensic settings that contain nucleic acids are also within the intended meaning of the term biological sample.

The term “buffer,” as used herein, refers to aqueous solutions or compositions that resist changes in pH when acids or bases are added to the solution or composition. This resistance to pH change is due to the buffering properties of such solutions. Thus, solutions or compositions exhibiting buffering activity are referred to as buffers or buffer solutions. Buffers generally do not have an unlimited ability to maintain the pH of a solution or composition. Rather, they are typically able to maintain the pH within certain ranges, for example between pH 5 and pH 7. Typically, buffers are able to maintain the pH within one log above and below their pK_{A}. See, generally, C. Mohan, Buffers, A guide for the preparation and use of buffers in biological systems, CALBIOCHEM®, 1999. Exemplary buffers include, but are not limited to, MES ([2-(N-Morphilino)ethanesulfonic acid]), ADA (N-2-Acetamido-2-iminodiacetic acid), and Tris ([tris (Hydroxymethyl)aminomethane]; also known as Trizma); Bis-Tris; ACES; PIPES; MOPS; and the like. Buffers and buffer solutions are typically made from buffer salts. Thus, for example but not as a limitation, to make a MES buffer one would use 2-(N-Morphilino)ethanesulfonic acid (or salts thereof); to make Tris buffer one would use Trizma base (or salts thereof) or Trizma HCl (or salts thereof), as appropriate; and so forth. Buffer solutions and buffer salts are commercially available from numerous sources, such as Sigma-Aldrich (St. Louis, Mo.), Fluka (Milwaukee, Wis.), and CALBIOCHEM (La Jolla, Calif.).

Buffers that maintain the pH within a certain pH range, for example, between pH 5 and pH 7, and similar terms used herein, are intended to encompass any buffer that exhibits buffering action at any pH within the stated pH range. Thus, the term buffer encompasses solutions and compositions that do not exhibit buffering capacity within the entire stated range, as well as solutions and compositions with buffering capacity that extend beyond the stated range. For example, solution A may exhibit buffering capacity between pH 5.2 and 6.7, solution B may exhibit buffering capacity between 6.0 and 8.0. For purposes of this invention, both of those solutions would be considered buffers that maintain the pH within the range of pH 5.0 to pH 7.0. The skilled artisan will be able to identify an appropriate buffer for maintaining the pH within a specified range using a buffer table. Buffer tables can be found in, among other places, the CALBIOCHEM 2000-2001 General Catalog at pages 81-82, the Sigma 2000-2001 Biochemicals and Reagents for Life Science Research Catalog at page 1873, and C. Mohan, Buffers, A guide for the preparation and use of buffers in biological systems, CALBIOCHEM®, 1999, at pages 18-21.

A “cationic detergent” has a positively charged group under the conditions examined. Typically, cationic detergents may contain quaternary amines or tertiary amines. However, at the proper pH, cationic detergents can contain primary or secondary amines. Exemplary quaternary amine detergents include, but are not limited to, cetylpyridinium chloride, cetyl trimethyl ammonium bromide (CTAB; Calbiochem #B22633 or Aldrich #85582-0), cetyl trimethyl ammonium chloride (CTACl; Aldrich #29273-7), dodecyl trimethyl ammonium bromide (Sigma #D-8638), octyl trimethyl ammonium bromide, tetradecyl trimethyl ammonium bromide, octadecylyl trimethyl ammonium bromide, stearoalkonium chloride, olealkonium chloride, cetrimonium chloride, alkyl trimethyl ammonium methosulfate, palmitamidopropyl trimethyl chloride, quaternium 84 (Mackernium NLE; McIntyre Group, Ltd.), wheat lipid epoxide (Mackernium WLE; McIntyre Group, Ltd.), and the like. Exemplary ternary amine detergents include, but are not limited to, octyldimethylamine, decyldimethylamine, dodecyldimethylamine, tetradecyidimethylamine, hexadecyldimethylamine, octyldecyldimethylamine, octyldecylmethylamine, didecylmethylamine, dodecylmethylamine, triacetylammonium chloride, cetrimonium chloride, alkyl dimethyl benzyl ammonium chloride, and the like.

The term “chaotrope” as used herein, refers to a substance that causes disorder in a protein or nucleic acid by, for example, but not limited to, altering the secondary, tertiary, or quaternary structure of a protein or a nucleic acid while leaving the primary structure intact. Exemplary chaotropes include, but are not limited to, guanidine hydrochloride (GuHCl), guanidinium thiocyanate (GuSCN), sodium thiocyanate (KSCN), sodium iodide, sodium perchlorate, urea, and the like. A typical anionic chaotropic series, shown in order of decreasing chaotropic strength, includes: CCl₃COO⁻>>CNS⁻>CF₃COO⁻>ClO₄>I⁻>CH₃COO—>Br⁻, Cl⁻, or CHO₂⁻. Descriptions of chaotropes and chaotropic salts can be found in, among other places, K. Hamaguchi et al., Proc. Natl. Acad. Sci. 62: 1129-1136, 1962; The Effect Of Electrolytes On The Stability Of The Deoxyribonucleate Helix, J. Amer. Chem. Soc. 84: 1329-1338; U.S. Patent Application Publication No. US 2002/0177139; and U.S. Pat. No. 5,234,809.

The term “nucleic acid,” as used herein, refers to a polymer of ribonucleosides or deoxyribonucleosides that are covalently bonded, typically by phosphodiester linkages between subunits, but in some cases by phosphorothioates, methylphosphonates, and the like. Such nucleic acids include, but are not limited to, gDNA; hnRNA; mRNA; noncoding RNA (ncRNA), including but not limited to rRNA, tRNA, mRNA (micro RNA), siRNA (small interfering RNA), snORNA (small nucleolar RNA), snRNA (small nuclear RNA) and stRNA (small temporal RNA); fragmented nucleic acid; nucleic acid obtained from subcellular organelles such as mitochondria or chloroplasts; and nucleic acid obtained from microorganisms, parasites, or DNA or RNA viruses that may be present in a biological sample. Synthetic nucleic acid sequences, that may or may not include include nucleotide analogs, that are added or “spiked” into a biological sample are also within the scope of the invention. Discussions of nucleic acids may be found in, among other places, Current Protocols in Nucleic Acid Chemistry, S. Beaucage, D. Bergstrom, G. Glick, and R. Jones, eds., John Wiley & Sons, 1999 including updates through August 2003; S. Verma and F. Eckstein, Ann. Rev. Biochem., 67:99-134, 1998; S. Buckingham, Horizon Symposia, Understanding the RNAissance, Nature Publishing Group, May 2003 at pages 1-3; S. Eddy, Nature Rev. Genetics 2:919-29, 2001; and Nucleic Acids in Chemistry and Biology, 2d ed., G. Blackburn and M. Gait, eds., Oxford University Press, 1996.

The term “or combinations thereof” as used herein, refers to all permutations and combinations of the items on the list preceding the term. For example, the term “A, B, C, or combinations thereof” is intended to include at least one of: A, B, C, AB, AC, BC, ABC; and if order is important in a particular context, also BA, CA, CB, CBA, and BCA. Continuing with this example, expressly included are combinations that include repeats of one or more item or term, such as BB, AAA, MB, BBC, AAABCCCC, CBBAAA, CAABBB, and so forth.

As used herein, the term “protease” refers to an enzyme that catalyzes the cleavage of peptide bonds, e.g., in proteins, polypeptides, oligopeptides, and peptides (collectively “peptides”). Exemplary proteases include, but are not limited to, subtilisins, subtilases, alkaline serine proteases, and the like. Subtilases are a family of serine proteases, i.e., enzymes with a serine residue in their active site. Subtilases are found in prokaryotic and eukaryotic organisms, such as bacteria, fungi, yeast, and higher organisms. Subtilisins are bacterial serine proteases that have broad substrate specificities. Subtilisins are relatively resistant to denaturation by chaotropes, such as urea and guanidine hydrochloride, and anionic detergents, such as sodium dodecyl sulfate. Exemplary subtilisins include, but are not limited to: Proteinase K; Proteinase R; Proteinase T (isolated from Tritirachium album Limber); Subtilisin DY, Carlsberg, also referred to as Subtilisin, Subtilisin A, Subtilopeptidase A, or Alcalase Novo; BPN′, also referred to as Nagarse proteinase, Nagarse, or Subtilopeptidase C; Novo, also referred to as Bacterial proteinase Novo, Subtilisin B, or Subtilopeptidase B; mesentericopeptidase; Thermitase; and the like. Discussions of subtilases, subtilisins, Proteinase K, and other proteases may be found, among other places, in Genov et al., Int. J. Peptide Protein Res. 45:391400, 1995; Narhi and Arakawa Biochimica et Biophysica Acta 990:144-149, 1989; Dixon and Webb, Enzymes, 3d Edition, 1979, Academic Press, New York, N.Y.; and Creighton, Proteins: Structures and Molecular Principles, 1984, W. H. Freeman and Co., New York, N.Y.

Solid phase materials, also referred to as solid phases or solid phase supports, that are capable of binding nucleic acids that are present in or are released from a biological sample, include a variety of materials that are capable of binding nucleic acids under suitable conditions. Exemplary solid phase components include, but are not limited to, compounds comprising silica, including but not limited to, silica particles, silicon dioxide, diatomaceous earth, glass, alkylsilica, aluminum silicate, and borosilicate; nitrocellulose; diazotized paper; hydroxyapatite (also refered to as hydroxylapatite); nylon; metal oxides; zirconia; alumina; diethylaminoethyl- and triethylaminoethyl-derivatized supports (Chromegabond SAX, LiChrosorb-AN, Nucleosil SB, Partisil SAX, RSL Anion, Vydac TP Anion, Zorbax SAX, Nucleosil Nme2, Aminex A-series, Chromex, and Hamilton HA lonex SB, DEAE Sepharose, QAE Sepharose); hydrophobic chromatography resins (such as phenyl- or octyl Sepharose); and the like. The term solid phase is not intended to imply any limitation regarding form. Thus, the term solid phase encompasses appropriate materials that porous or non-porous; permeable or impermeable; including but not limited to membranes, filters, sheets, particles, beads, gels, powders, fibers, and the like. In certain embodiments, the combination is exposed to at least one solid phase and nucleic acid binds to the solid phase. The skilled artisan will appreciate that the term exposing is not limiting and includes for example, but not limited to, comprises contacting, combining, adding, and the like.

The term “zwitterionic compound” as used herein, refers to a substance that, under appropriate conditions, is capable of simultaneously carrying both positive and negative charges on the same group of atoms or compound, and that typically have a net zero charge. Exemplary zwitterionic compounds of the invention include zwitterionic detergents, including but not limited to sulfobetaines; and the non-detergent zwitterions, including but not limited to non-detergent sulfobetaines (NDSBs), and the like. Zwitterionic detergents and non-detergent zwitterions typically differ by the nature of the groups attached to their zwitterion portion. For example, but without limitation, zwitterionic detergents tend to have large nonpolar aliphatic side chains, while non-detergent zwitterions have side chains that typically are not strongly hydrophobic. Zwitterionic compounds typically have some properties of both nonionic and ionic detergents. For example, they typically fail to bind ion-exchange resins and lack electrophoretic mobility, like the nonionic detergents; but like ionic detergents, they efficiently disrupt many protein-protein interactions. Exemplary zwitterionic detergents include N,N-bis(3-D-Gluconamidopropyl)cholamide (BigCHAP), 3-(3-Cholamidopropyl)dimethylammonio]-1-propanesulfonate (CHAPS), 3-[(3-Cholamidopropyl)dimethylammonio]-2-hydroxy-1-propanesulfonate (CHAPSO), N-dodecyl-N,N-(dimethylammonio)butyrate (DDMAB), DDMAU, N-Dodecyl-N,N-dimethylglycine (EMPIGEN BB), Lauryldimethylamine N-oxide (LDAO), n-Octyl-N,N-dimethyl-3-ammonio-1-propanesulfonate, n-Decyl-N,N-dimethyl-3-ammonio-1-propanesulfonate, n-Dodecyl-N,N-dimethyl-3-ammonio-1-propanesulfonate, n-Tetrtadecyl-N,N-dimethyl-3-ammonio-1-propanesulfonate, n-Hexadecyl-N,N-dimethyl-3-ammonio-1-propanesulfonate, n-Octadecyl-N,N-dimethyl-3-ammonio-1-propanesulfonate, 3-(Dodecyldimethylammonio)propanesulfonate, 3-(N,N-Dimethylpalmitylammonio)propanesulfonate, amidosulfobetaines such as ASB-14, ASB-16 (both from CALBIOCHEM, La Jolla, Calif.), and the like. Some synthetic zwitterionic detergents are also known as sulfobetaines.

The non-detergent sulfobetaines, or NDSBs, are typically unable to aggregate and thus do not form micelles. NDSBs typically have a short hydrophobic group and a sulfobetaine hydrophilic group, while zwitterionic detergents typically have a quaternary ammonium ion and a sulfonate group, often with zero net charge. Exemplary non-detergent sulfobetaines include NDSB-195, NDSB-201, NDSB-211, NDSB-221, NDSB-256, and the like. Further descriptions of zwitterionic detergents, NDSBs, and sulfobetaines may be found in, among other places, CALBIOCHEM General Catalog 2002-2003; S. Bhairi, A Guide to the Properties and Uses of Detergents in Biology and Chemistry, CALBIOCHEM®; Hjelmeland, Proc Natl Acad Sci USA, 77(11):6368-70,1980; and Neugebauer, Methods Enzymol. 182:239-53 (1990).

Exemplary Embodiments of the Invention:

The present invention is directed to compositions, methods, and kits for obtaining and binding and for isolating nucleic acid from biological samples, frequently body fluids, swabs, tissues, or tissue culture cells. Typically the nucleic acid being obtained is initially present within cells in the sample, but not always (see, e.g., Circulating Nucleic Acids in Plasma and Serum II, Y. Lo, R. Chu, and P. Johnson, eds., Annals of the New York Academy of Sciences, vol. 945, 2001). The compositions, methods, and kits of the invention reduce the time needed for sample preparation, decrease potential safety risks posed by multi-step procedures that require repeated sample manipulation, and provide high integrity (i.e., minimally degraded) nucleic acid, including but not limited to high molecular weight gDNA. Additionally, the isolated nucleic acid according to the inventive methods and kits is of high quality in terms of the absence of contaminants that typically inhibit PCR or other downstream enzymatic reactions.

In certain embodiments, the methods employ and the kits comprise at least one protease and at least one solid phase. In certain embodiments, these methods and kits further comprise at least one zwitterionic compound, at least one first chaotrope, at least one second chaotrope, at least one non-ionic detergent, at least one cationic detergent, or combinations thereof.

In certain embodiments, the methods employ and the kits comprise at least one first chaotrope and at least one solid phase. In certain embodiments, these methods and kits further comprise at least one zwitterionic compound, at least one second chaotrope, at least one non-ionic detergent, at least one cationic detergent, at least one protease, or combinations thereof.

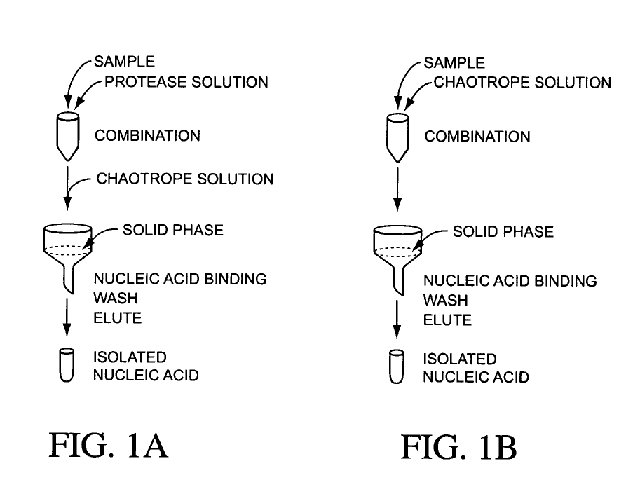

In certain embodiments, the sample is combined with at least one protease solution comprising at least one protease to form a combination, see, e.g., FIG. 1A. In certain embodiments, the protease solution further comprises at least one chaotrope, at least one zwitterionic compound, or at least one chaotrope and at least one zwitterionic compound. In certain embodiments, at least one chaotrope is added separately, at least zwitterionic compound is added separately, of both at least one chaotrope and at least zwitterionic compound are added separately. In certain embodiments, the protease solution further comprises at least one buffer solution and/or buffer salt. In certain embodiments, the protease solution is incubated under conditions appropriate for nucleic acids to be released from the sample. In certain embodiments, nucleic acids are released immediately or almost immediately. Thus, in certain embodiments, the incubation time may be extremely short or even instantaneous. The combination is then exposed to at least one solid phase and at least one nucleic acid binds to the solid phase to form at least one Complex. Optionally, the Complex is separated from unbound material of the combination. In certain embodiments, the bound nucleic acid is isolated from the Complex, typically by elution.

In certain embodiments, the sample is combined with at least one first chaotrope and at least one zwitterionic compound to form a combination. In certain embodiments, the sample is combined with a chaotrope solution comprising at least one chaotrope and at least one zwitterionic compound, see, e.g., FIG. 1B. In certain embodiments, the combination is incubated under conditions appropriate for nucleic acids to be released. In certain combinations, nucleic acids are released immediately or almost immediately. Thus, in certain embodiments, the incubation time may be extremely short or even instantaneous. The combination is then exposed to at least one solid phase and at least one nucleic acid binds to the solid phase to form at least one Complex. The Complex is separated from unbound material of the combination. In certain embodiments, the bound nucleic acid is isolated from the Complex, typically by elution.

In certain embodiments the solid phase is in the form of a filter, filter paper, particles or beads, and the like, and may or may not comprise additional components. In certain embodiments, the solid phase may be present in a support, vessel, container, or some other form of inert or partially inert material. During and/or after Complex formation, the unbound material from the combination may be separated by filtration, percolation, sedimentation, centrifugation, or the like. The skilled artisan will appreciate that separating may comprise applying at least one of following forces: gravity; vacuum; centrifugal; centripetal; pressure, including but not limited to, pneumatic pressure (e.g., compressed air or other gas), hydraulic pressure, osmotic pressure; or similar forces, including combinations thereof.

In certain embodiments, separating further comprises washing the Complexes, typically bound on or in the solid phase. In certain embodiments, washing comprises a variety of solutions, buffers, and the like, with and without surfactants. In certain embodiments, a wash solution comprises a buffer containing at least one chaotrope. In certain embodiments a wash solution comprises at least one alcohol. Exemplary alcohols for wash solutions include methanol, ethanol, propanol, butanol, pentanol, and the like; straight-chain, cyclic, and branched forms of alcohols; as well as absolute or denatured alcohols. In certain embodiments, the nucleic acid to be isolated is eluted from the Complex using a solution comprising at least one alkaline component, for example but not limited to a buffer solution with a pH greater than 10, a buffer solution with a pH greater than 9, or a buffer solution with a pH greater than 8. Exemplary alkaline solutions comprise, but are not limited to sodium hydroxide (NaOH), potassium hydroxide (KOH), bicine, tricine, AMPS, AMPSO, and the like. In certain embodiments, the nucleic acid to be isolated is eluted from the Complex using a low ionic strength solution, such as Tris-EDTA (TE) buffer, distilled deionized water, or the like.

In certain embodiments, the methods and kits comprise at least one first chaotrope. In certain embodiments, the methods and kits further comprise at least one second chaotrope. In certain embodiments, the at least one first chaotrope and the at least one second chaotrope may be the same or different. Thus, for example but without limitation, in certain embodiments the at least one first chaotrope may comprise GuHCl and the at least one second chaotrope may comprise GuSCN, or vice versa; while in other embodiments, the at least one first chaotrope and the at least one second chaotrope may both comprise, for example GuSCN.

In certain embodiments of the invention, the at least one first chaotrope, the at least one second chaotrope, or both the at least one first chaotrope and the at least one second chaotrope comprise at least one of: guanidine hydrochloride (GuHCl); guanidinium thiocyanate (GuSCN); urea; sodium bromide (NaBr); sodium iodide (NaI); sodium perchlorate (NaClO₄); sodium thiocyanate (NaSCN); lithium chloride (LiCl); lithium bromide (LiBr); lithium iodide (LiI); potassium iodide (KI); potassium chloride (KCl); potassium bromide (KBr); tetrabutyl ammonium bromide; tetrabutyl ammonium chloride; tetrabutyl ammonium iodide; tetrapropyl ammonium bromide; tetrapropyl ammonium chloride; tetrapropyl ammonium iodide; thiourea (NH₂CSNH₂), also known as sulfourea or thiocarbamide; potassium thiocyanate (KSCN); or combinations thereof.

In certain embodiments, the methods and kits of the invention further comprise at least one zwitterionic compound. Such zwitterionic compounds typically comprise, for example but without limitation, zwitterionic detergents, sulfobetaines, non-detergent sulfobetaines, or combinations thereof. Applicants have observed that methods of the invention comprising at least one zwitterionic compound provide increased yields and increased purity of high integrity gDNA from biological samples, such as whole blood samples and increased consistency between samples. In certain embodiments, wherein separating comprises filtration, the presence of at least one zwitterionic compound in the combination results in reduced clogging of the solid phase.

In certain embodiments, the at least one zwitterionic compound comprises at least one of n-Octyl-N,N-dimethyl-3-ammonio-1-propanesulfonate; n-Decyl-N,N-dimethyl-3-ammonio-1-propanesulfonate; n-Dodecyl-N,N-dimethyl-3-ammonio-1-propanesulfonate; n-Tetradecyl-N,N-dimethyl-3-ammonio-1-propanesulfonate; n-Hexadecyl-N,N-dimethyl-3-ammonio-1-propanesulfonate; n-Octadecyl-N,N-dimethyl-3-ammonio-1-propanesulfonate; Lauryldimethylamine N-oxide (LDAO™); 3-[(3-Cholamidopropyl)dimethylammonio]-1-propanesulfonate (CHAPS™); 3-[(3-Cholamidopropyl)dimethylammonio]-2-hydroxy-1-propanesulfonate (CHAPSO™); N,N-bis(3-D-Gluconamidopropyl)cholamide (BigCHAP); N-Dodecyl-N,N-dimethylglycine (Empigen BB™); N-dodecyl-N,N-(dimethylammonio)butyrate (DDMAB™); amidosulfobetaine detergents such as ASB-14™ and ASB-16™, commercially available from CALBIOCHEM; dodecylbetaine; lauraminopropyl betaine; dipalmitoylphosphatidylcholine (lecithin); N-dodecyl-N,N-(dimethylammonio)undecanoate (DDMAU); n-decyl-N,N-dimethylglycine; N-octylphosphocholine; N-nonylphosphocholine; N-decylphosphocholine; N-dodecylphosphocholine; N-tetradecylphosphocholine; N-hexadecylphosphocholine; Octyl phospho-n-methylethanolamine; Decyl phospho-n-methylethanolamine; Dodecyl phospho-n-methylethanolamine; Cyclohexylethylphosphocholine; Cyclohexylpropylphosphocholine; Cyclohexylbutylphosphocholine, dimethylethylammonium propane sulfonate; 3-(1-pyridino)-1-propane sulfonate; dimethyl-(2-hydroxyethyl)-(3-sulfopropyl)-ammonium; 3-(1-methylpiperidinium)-1-sulfonate; dimethylbenzylammonium propane sulfonate; dimethylethyl-(3-sulfopropyl)-ammonium; N-phenyl-methyl-N,N-dimethylammonium-propane-sulfonate; 3-(Decyidimethylammonio)propanesulfonate; 3-(Dodecyldimethylammonio)propanesulfonate; 3-(N,N-Dimethylmyristylammonio)propanesulfonate; 3-(N,N-Dimethyloctylammonio)propanesulfonate; 3-(N,N-Dimethylpalmitylammonio)propanesulfonate; and the like. Zwitterionic compounds are commercially available from numerous sources, including but not limited to, CALBIOCHEM (La Jolla, Calif.), Sigma-Aldrich (St. Louis, Mo.), and A.G. Scientific (San Diego, Calif.).

In certain embodiments, methods and kits of the invention further comprise, at least one DNase inhibitor. For example, many endogenous DNases are inhibited by divalent cation chelators, such as EDTA (ethylenediaminetetraacetic acid), EGTA ([ethyleneglycol-bis(β-aminoethyl)-N,N,N′,N′-tetraacetic acid]), DTPA (diethylenetriaminepentaacetic acid), and the like.

In certain embodiments, the at least one cationic detergent used in the methods and kits has the general chemical formula: 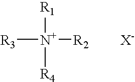
(N⁺R₁R₂R₃R₄:X⁻), where the cation moieties R₁, R₂, R₃, and R₄ independently may be: —H, an alkyl group containing up to 20 carbon atoms, or an aryl group containing between 6 and 26 carbon atoms; wherein at least one of R₁, R₂, R₃, or R₄ contains an alkyl group of at least 6 carbons; and where X⁻ is an anion. For example, the cationic surfactant may be an alkyltrimethyl ammonium salt, where R₁, R₂, and R₃ are methyl groups, and R₄ is an alkyl group comprising 6, 8, 10, 12, 14, 16, or 18 carbon atoms. The cationic subcomponent (N+R₁R₂R₃R₄) of the alkyltrimethyl ammonium salt might be (without limitation) a cetyltrimethylammonium group, a hexadecyltrimethylammonium group, a tetradecyltrimethylammonium group, a dodecyltrimethylammonium group, a lauryl trimethylammonium group, or the like. The anionic subcomponent (X⁻) of this exemplary alkyltrimethyl ammonium salt might be (without limitation) any of the following ions: bromide, chloride, iodide, hydroxide, nitrate, sulfate, phosphate, formate, acetate, propionate, oxalate, malonate, succinate, or citrate. In certain embodiments, the cationic surfactant is a benzyldimethyl-n-alkylammonium salt, comprising the same group of anions.

In certain embodiments, the at least one cationic detergent comprises at least one alkyltrimethyl ammonium salt wherein the cation is selected from the group consisting of: cetyltrimethylammonium; hexadecyltrimethylammonium; tetradecyltrimethylammonium; dodecyltrimethylammonium; or lauryl trimethylammonium. In certain embodiments, the anion (X⁻) of the at least one cationic detergent is selected from the group consisting of: bromide; chloride; iodide; hydroxide; nitrate; sulfate; phosphate; formate; acetate; propionate; oxalate; malonate; succinate; or citrate.

In certain embodiments, the at least one cationic detergent comprises at least one of: cetyltrimethylammonium bromide (CTAB); cetyltrimethylammonium chloride (CTACl); tetradecyltrimethylammonium bromide (TTAB); tetradecyltrimethylammonium chloride (TTACl); dodecyltrimethylammonium bromide (DTAB); dodecyltrimethylammonium chloride (DTACl); dodecylethyldimethylammonium bromide (DEDTAB); decyltrimethylammonium bromide (D₁₀TAB); or dodecyltriphenylphosphonium bromide (DTPB).

The invention also provides kits designed for isolating nucleic acids from biological samples. In certain embodiments, kits comprise at least one chaotrope and at least one solid phase. In certain embodiments, kits comprise at least one protease and at least one solid phase. In certain embodiments kits further comprise at least one protease, at least one first chaotrope, at least one second chaotrope, at least one non-ionic detergent, at least one cationic detergent, at least one wash solution, at least one wash buffer, at least one alcohol, at least one elution solution, at least one elution buffer, at least one buffer salt, at least one DNase inhibitor, or combinations thereof. In certain embodiments, the kits preferably contain components in pre-measured unit amounts to minimize the measurements by end-users. In certain embodiments, the kits preferably include instructions for performing one or more methods of the invention. Preferably, the kit components are optimized to operate in conjunction with one another.

The invention, having been described above, may be better understood by reference to examples. The following examples are intended for illustration purposes only, and should not be construed as limiting the scope of the invention in any way.

### EXAMPLE 1

### Effect of Detergents on Nucleic Acid Binding to Solid Phase

Detergents are commonly used in sample preparation chemistries to prevent nonspecific binding of proteins. Some detergents, however inhibit the binding of nucleic acid to the solid phase. The effect of various nonionic and zwitterionic detergents on the binding of protein-free nucleic acid to a GF/B glass fiber filter (Whatman Biosciences, Cat # 1821-150), in the presence of a chaotrope was evaluated. Seventeen samples, containing four micrograms (μg) of purified, partially sheared calf thymus gDNA in chaotrope solution comprising 4M GuSCN, 50 mM MES, pH 6, and 2% detergent (one of the seventeen shown in Table 1) were filtered in parallel through a solid phase comprising GF/B glass fiber filters installed in a 96-well universal purification tray (Applied Biosystems P/N 4307633). A control sample, containing no detergent, was also included.

TABLE 1

| Detergent name | Detergent type |
|---|---|
| CHAPS ® | Zwitterionic |
| ZWITTERGENT ® 3-14 | Zwitterionic |
| (n-Tetradecyl-N,N-dimethyl-3- | |
| ammonio-1-propanesulfonate) | |
| ZWITTERGENT ® 3-16 | Zwitterionic |
| (n-Hexadecyl-N,N-dimethyl-3- | |
| ammonio-1-propanesulfonate) | |
| TERGITOL ® TMN-6 | Non-ionic |
| TERGITOL ® Min Foam IX | Non-ionic |
| TERGITOL ® NP-9 | Non-ionic |
| TERGITOL ® NP-10 | Non-ionic |
| IGEPAL (NP-40) | Non-ionic |
| SPAN ® 20 | Non-ionic |
| BRIJ ® 30 | Non-ionic |
| Triton ® X-100 | Non-ionic |
| Tween ® 20 | Non-ionic |
| Tween ® 21 | Non-ionic |
| Tween ® 40 | Non-ionic |
| Tween ® 60 | Non-ionic |
| Tween ® 80 | Non-ionic |
| Tween ® 85 | Non-ionic |

The purification tray was mounted on the ABI PRISM 6100 Nucleic Acid PrepStation (Applied Biosystems, Foster City, Calif.) and the samples were allowed to pass through the glass fiber filter under 2.4 psi vacuum. After sample evacuation, the filters comprising bound Complexes was washed with 90% ethyl alcohol solution (Sigma No. 7023). A one hundred microliter aliquot of 0.01N NaOH was added to each filter and incubated for three minutes at ambient temperature to elute the DNA from the Complexes. The DNA-containing eluate was evacuated and collected into the 96-well sample archive tray (ABI PRISM 4306737, Applied Biosystems), followed by 100 μL 15 mM Tris-HCl, 1 mM EDTA, pH 7.0. The second eluate was collected into the archive tray containing the first eluate. Elution with high pH solution was found to increase the efficiency of gDNA elution from the filter-bound Complexes. After pooling the first and second eluates, the pH of the combined eluate was approximately pH 8.

The quantity of isolated nucleic acid was determined using a UV/V is Spectrophotometer (Hewlett Packard, Model 8453). For quantitation purposes, it was assumed in all of the examples herein that the material that absorbed UV light at a wavelength of 260 nm was DNA. Thus, an extinction coefficient of 1 OD₂₆₀=50 μg/mL was used throughout.

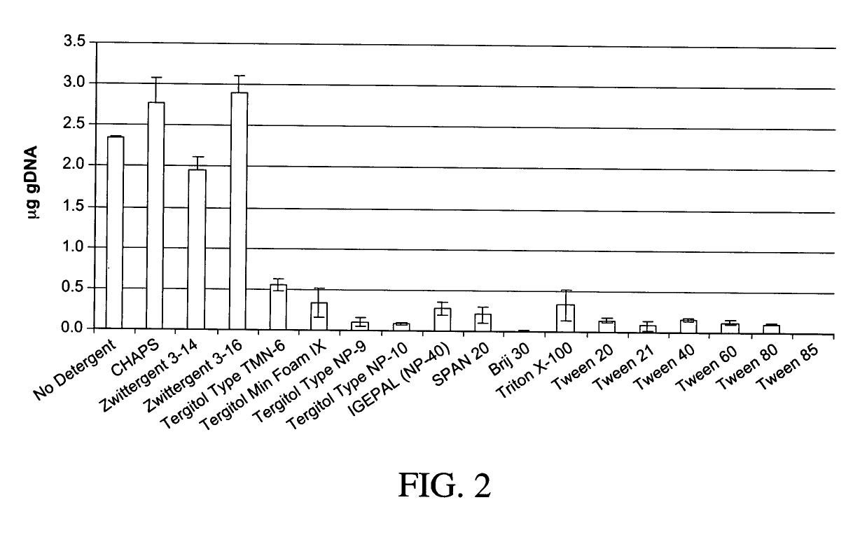

As seen in FIG. 2, under the conditions tested, none of the zwitterionic compounds significantly inhibited recovery of nucleic acid, while the recovery was greatly decreased with all of the non-ionic detergents, as determined by the yield of isolated nucleic acid. However, the nonionic detergents evaluated under these conditions provided lower yields of nucleic acids.

### EXAMPLE 2

### The Effect of Surfactant on Filtration of Protease-Treated Whole Blood Sample

Two sets of twenty microfuge tubes (1.5 mL), each containing 200 microliters (μL) of protease solution (100 mM Tris buffer, pH 8.0 (Sigma T-3038) and 2 mg Proteinase K (Ambion Cat. # 2548)), were prepared in parallel. To each tube 150 μL whole fresh blood was added, then the tubes were incubated in a 55° C. water bath for 15 minutes. Six hundred microliters of chaotrope solution (5M GuSCN, 20 mM EDTA, 60 mM MES buffer, pH 6.0, and 2% of one of the surfactants listed in Table 2) was added to each tube with vigorous mixing. The combinations were transferred into individual wells of a GF/B glass fiber filter tray assembly (Whatman Biosciences, Cat # 1821-150; Applied Biosystems P/N 4307633) mounted on an ABI PRISM 6100 Nucleic Acid PrepStation. The combinations were passed through the filters under 2.4 psi vacuum and Complexes formed. Each glass fiber filter was visually inspected to determine whether filter clogging occurred during separation (see Table 2).

TABLE 2

| Surfactant | Result |
|---|---|
| Anionic Detergent | |
| Hexanoic acid | Clogged |
| Octanoic acid | Clogged |
| Decanoic acid | Not Clogged |
| Taurodeoxycholic acid | Clogged |
| Hexane sulfonic acid | Clogged |
| Octyl sulfate | Clogged |
| Sodium laurylsarcosine | Clogged |
| Nonionic detergent | |
| Tergitol TMN-6 | Clogged |
| Triton X-100 | Clogged |
| Tween 20 | Clogged |
| Zwitterionic compound | |
| Empigen BB | Not Clogged |
| DDMAB | Not Clogged |
| CHAPS | Clogged |
| LDAO | Not Clogged |
| Zwittergent 3-12 | Not Clogged |
| Zwittergent 3-14 | Not Clogged |
| Zwittergent 3-16 | Not Clogged |
| NDSB-195 | Clogged |
| NDSB-201 | Clogged |
| NDSB-256 | Clogged |

### EXAMPLE 3

### Effect of Zwitterionic Compounds on the Isolation of Nucleic Acid

Aliquots of 150 μL fresh whole blood were placed in 1.5 mL microfuge tubes containing 200 μL of protease solution (100 mM Tris buffer, pH 8.0 (Sigma T-3038) and 2 mg Proteinase K (Ambion Cat. # 2548). The tubes were incubated in a 55° C. water bath for 15 minutes, then combined with 600 μL of chaotrope solution (5M GuSCN, 20 mM EDTA, 60 mM MES buffer, pH 6.0, and 2% of one of the surfactants listed in Table 3 with vigorous mixing. The combinations were transferred into individual wells of a GF/B glass fiber filter tray (Whatman Biosciences, Cat # 1821-150) mounted on the ABI PRISM 6100 Nucleic Acid PrepStation and evacuated under 2.4 psi vacuum.

TABLE 3

| Zwitterionic Compound | Result |
|---|---|
| CHAPS | Clogged |
| Empigen BB | Not Clogged |
| LDAO | Not Clogged |
| DDMAB | Not Clogged |
| Zwittergent 3-08 | Clogged |
| Zwittergent 3-12 | Not Clogged |
| Zwittergent 3-14 | Not Clogged |
| Zwittergent 3-16 | Not Clogged |
| Tween 20 (nonionic detergent) | Clogged |
| NDSB 195 | Clogged |
| NDSB 201 | Clogged |
| NDSB 256 | Clogged |

Following vacuum filtration of the combinations, the filters were visually inspected to evaluate clogging. As shown in Table 3, under these conditions, only combinations comprising zwitterionic detergents passed through the filter without clogging. Nucleic acids in the combination form Complexes with the solid phase (filters) and the remaining components of the combinations pass through the unclogged filters. The GF/B glass fiber filters were washed once with 650 μL of the chaotrope solution containing the appropriate test detergent, then three times with 650 μL 90% ethanol. The combinations from clogged filters were not processed further.

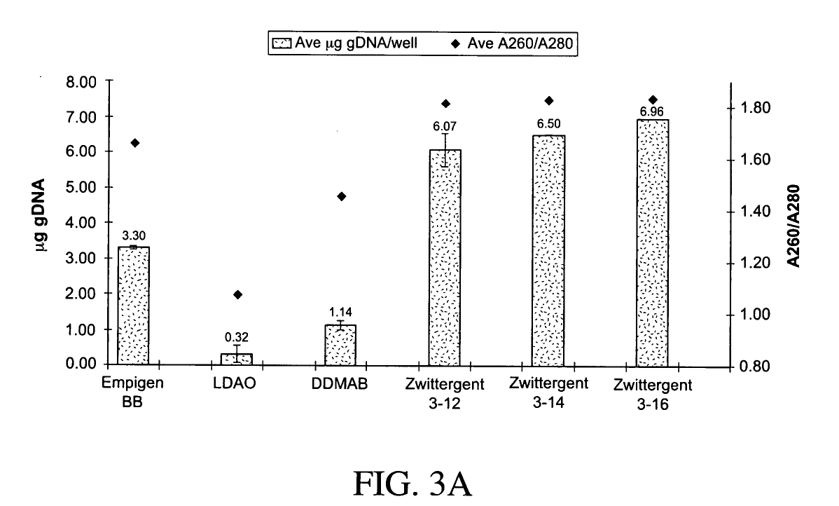

After washing, the glass fiber filters were allowed to dry for three minutes under 3 psi vacuum, then 100 μL 10 mM NaOH was added to each filter. Following a 3 minute incubation at ambient temperature, the nucleic acid was eluted from the filter-bound Complexes and the eluate was collected in a 96-well sample archive tray (ABI PRISM 4306737, Applied Biosystems). To neutralize the eluates, a 100 μL aliquot of 15 mM Tris-HCl, pH 7.0 was evacuated through the glass filter and collected in the archive tray with the corresponding eluate. The quantity and purity of the isolated nucleic acid was determined using a UV/V is Spectrophotometer, as described, and the results depicted graphically, as shown in FIG. 3A. Ten microliter aliquots of isolated nucleic acid were analyzed by gel electrophoresis to evaluate the quality of the recovered gDNA (see FIG. 3B).

### EXAMPLE 4

### Effect of Zwitterionic Compound Concentration on Nucleic Acid Isolation

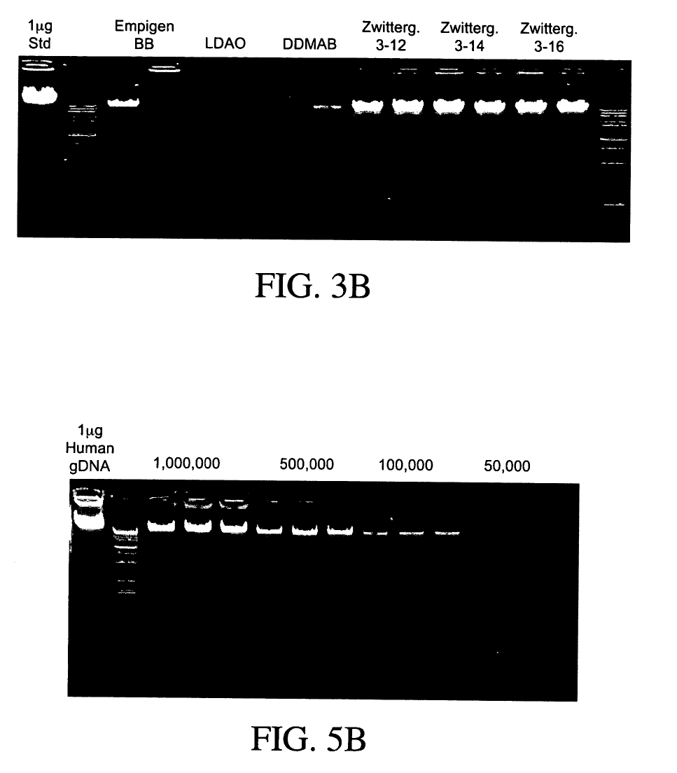

Ten parallel microfuge tubes were prepared containing 500 μL of chaotrope solution (5M GuSCN, 20 mM EDTA, 60 mM MES, pH 6.0, and varying amounts of Zwittergent 3-16 (0, 0.1, 0.5, 1.0, 1.5, 2.0, 2.5, 3.0, 3.5, or 4.0%, weight to volume)). To each tube, 200 μL fresh whole blood sample was added to form combinations. The combinations were transferred to GF/B glass fiber filters and vacuum filtered, as described in Example 3. The filters, comprising the bound Complexes, were washed once with chaotrope solution, then three times with 90% ethanol. The bound nucleic acid was eluted with 100 μL 10 mM NaOH, followed by 100 μL 15 mM Tris-HCl, pH 7.0 and the eluates were collected in a 96-well archive tray, as before. The yield of isolated nucleic acid in the eluate was determined using a UV/V is spectrophotometer (Hewlett Packard, Model No. 8453) and the results were depicted graphically, as shown in FIG. 4.

### EXAMPLE 5

### Isolation of Nucleic Acid from Swab Samples

Buccal swab samples were obtained in quintuplicate from seven donors. The swabs were placed in separate wells of a deep well plate (Applied Biosystems P/N 4308641), each containing 800 μL of a chaotrope solution (5 M GuSCN, 20 mM EDTA, 2% Zwittergent 3-16, 60 mM MES, pH 6.0), to form combinations. The swabs were soaked for two minutes at ambient temperature and each swab was briefly swirled during the incubation. A 450 μL aliquot of each combination was transferred to a separate well in a GF/B tray installed on the ABI PRISM 6100 Nucleic Acid PrepStation and vacuum filtered, as described in Example 3. The filters were washed three times with 90% ethanol, then eluted with 100 μL 10 mM NaOH, followed by 100 μL 15 mM Tris-HCl, pH 7.0 to neutralize the eluate collected in a 96-well sample archive tray (ABI PRISM 4306737). The yield of isolated nucleic acid was determined using 18S rDNA TaqMan Assay (Applied Biosystems P/N 4308329) on an ABI PRISM 7700 Sequence Detection System (Applied Biosystems). The results are shown in Table 4 below.

TABLE 4
Quantity (in μg) of Nucleic Acid Isolated From Buccal Swabs

| | Sample 1 | Sample 2 | Sample 3 | Sample 4 | Sample 5 |
|---|---|---|---|---|---|
| Donor 1 | 256 | 83 | 29 | 29 | 105 |
| Donor 2 | 9618 | 5805 | 5409 | 6208 | 6071 |
| Donor 3 | 14 | 15 | 62 | 45 | 58 |
| Donor 4 | 6575 | 83 | 197 | 223 | 1443 |
| Donor 5 | 35 | 9 | 10 | 45 | 64 |
| Donor 6 | 104 | 74 | 92 | 28 | 83 |
| Donor 7 | 65 | 25 | 65 | 44 | 45 |

### EXAMPLE 6

### Isolation of Nucleic Acid from Cultured Human Cells

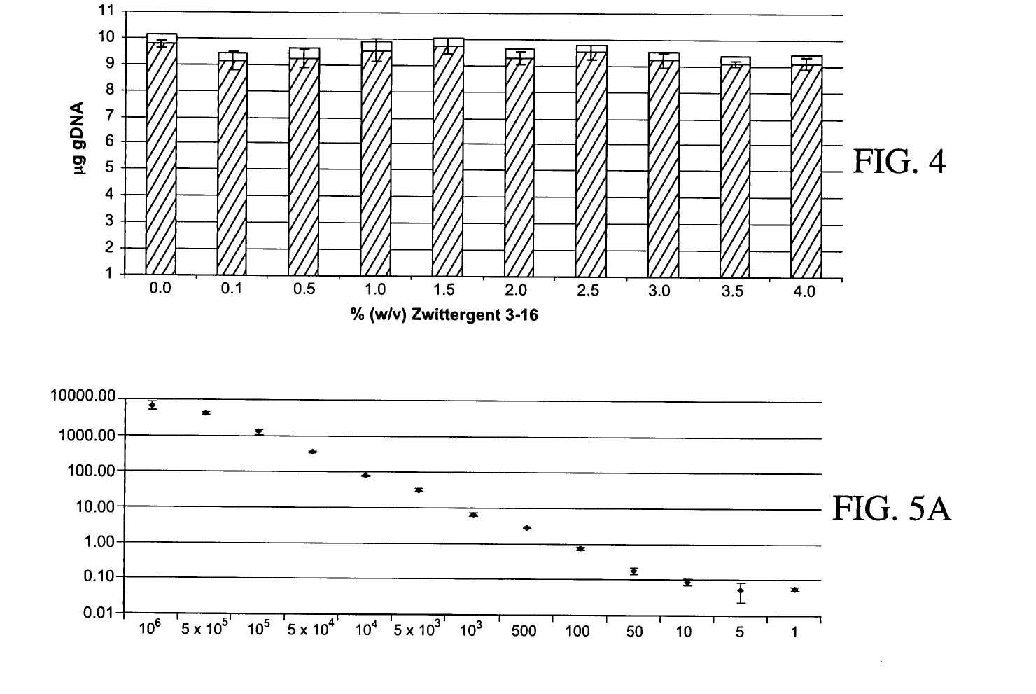

Human Raji cells, a B lymphocyte cell line obtained from a patient with Burkitt's lymphoma (ATCC CCL-86, American Type Culture Collection, Manassas, Va.), were grown in culture according to the cell propagation method and cell culture medium recommended by ATCC. The cells were centrifuged at 3000 rpm in an Eppendorf Model 5416 bench-top centrifuge and the resulting cell pellet was re-suspended in chilled phosphate-buffered saline (PBS; Sigma # P8357) to a final concentration of 1×10⁷ cells/mL. An aliquot of this cell suspension was diluted in chilled PBS to make a ten-fold dilution series ranging from 1×10⁷ cells/mL (undiluted) to 10 cells/mL. A two-fold dilution was made from each of the ten-fold dilutions, resulting in a total of 13 different cell dilutions (1×10⁷, 5×10⁶, 1×10⁶, 5×10⁵, 1×10⁵, 5×10⁴, 1×10⁴, 5000, 1000, 500, 100, 50 and 10 cells/mL). A 100 μL aliquot of each cell suspension was placed into individual wells of a deep well tray (Applied Biosystems P/N 4308641). Into each of the thirteen wells, 400 μL of a chaotrope solution (5M GuSCN, 50 mM MES, pH 6.0, 20 mM EDTA and 2% Zwittergent 3-16) was added with mixing, to form combinations. The combinations were transferred into individual wells of the 96-well GF/B filter tray installed on the ABI PRISM 6100 Nucleic Acid PrepStation containing a GF/B glass fiber filter and evacuated under 2.4 psi vacuum, as before. The filters, comprising the bound Complexes, were washed once with the chaotrope solution, three times with 90% ethanol, then 100 μL 0.01N NaOH was added to each of the washed wells. Following a three minute incubation at ambient temperature the eluate was evacuated and collected into a 96-well sample archive tray (ABI PRISM 4306737, Applied Biosystems). The first eluate was neutralized by evacuating 100 μL 15 mM Tris-HCl, pH 7.0 through the same filters and the second eluate was collected into the corresponding wells of the same sample archive tray. The quantity of nucleic acid isolated from each cell sample was measured using a UV/V is Spectrophotometer, as described, and the results depicted graphically, as shown in FIG. 5A. To evaluate the integrity of the isolated gDNA, 10 μL of neutralized eluate from cell dilutions containing 1×10⁶-5×10⁴ cells (three replicate eluates for each dilution point) were electrophoresed in triplicate on a 1% agarose gel, then stained with ethidium bromide using standard methodology, as shown in FIG. 5B.

### EXAMPLE 7

### Nucleic Acid Isolation Using Silica Particles

To evaluate the ability of silica particles to bind nucleic acids from a blood sample, five, seven, or ten milligrams of silica particles (P/N 2046, CERAC, Inc., Milwaukee, Wis.), respectively, were sandwiched between two polypropylene frits and installed in individual wells of a 96-well universal purification tray (Applied Biosystems P/N 4307633). Protease solution (1 mg proteinase K (Ambion), 100 mM Tris-HCl, pH 8.0 (Sigma)) was combined with 25, 50, or 100 μL whole blood sample in microfuge tubes. These tubes were incubated at 58° C. for 10 minutes, then 500 μL of chaotrope solution (5 M GuSCN, 50 mM EDTA pH 8.0, 200 mM Tris-HCl, pH 7.0 and 2% Zwittergent 3-16) was added with mixing to form combinations. These combinations were transferred to the wells of the silica particle-containing purification tray, described above, mounted on the ABI PRISM 6100 Nucleic Acid PrepStation. The wells were evacuated at 2.4 psi vacuum, washed once with chaotrope solution, then three times with 90% ethyl alcohol. The nucleic acid was eluted from the bound Complexes by adding 100 μL 0.01 N NaOH to each well, followed by 15 mM Tris-HCl, 1 mM EDTA pH 7.0, both under 2.4 psi vacuum. The isolated gDNA was collected in a 96-well sample archive tray (Applied Biosystems P/N 4306286).

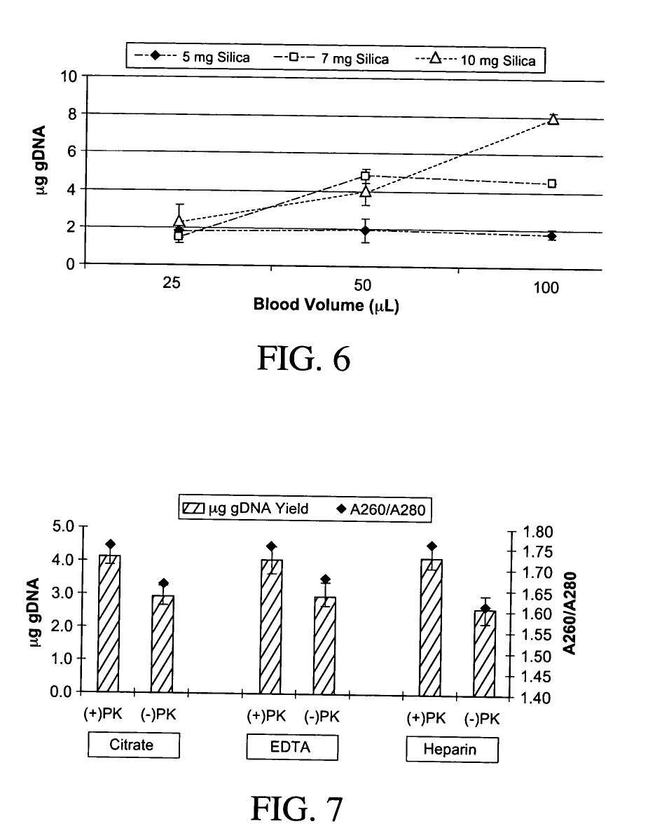

The yields of nucleic acid isolated from the three different volumes of whole blood sample using the three different quantities of silica particles were determined using a UV/V is Spectrophotometer as before, and the results shown graphically in FIG. 6. In this exemplary embodiment, with the 50 and 100 μL sample volumes, decreasing the quantity of silica particles resulted in decreasing amounts of isolated nucleic acid, suggesting that the binding capacity of the silica particles was saturated.

### EXAMPLE 8

### Isolation of DNA from Frozen Blood Sample with and Without Protease Treatment

To evaluate the effectiveness of protease digestion on previously frozen whole blood samples collected in citrate, EDTA and heparin anticoagulants, an experiment was performed to measure the yield and purity of nucleic acids isolated from such samples with and without protease solution. Two sets of nine microfuge tubes were prepared in parallel and 150 μL of thawed (I.e., previously frozen) whole human blood containing the anticoagulant citrate, EDTA and heparin was placed separately into each tube. Three replicates were prepared for each anticoagulant type. Into the protease (+) set of tubes 100 μL of a protease solution (300 ng proteinase K (Ambion), 0.4% CTAB (Sigma), 250 mM GuHCl (Sigma) and 125 mM Tris-HCl, pH 8.0 (Sigma)) was added to form digestion solutions. The tubes were incubated for 10 minutes in a 58° C. water bath. Then, 500 μL of a chaotrope solution (5 M GuSCN, 50 mM EDTA pH 8.0, 200 mM Tris-HCl, pH 7.0, 0.01% Antifoam and 2% Zwittergent 3-16) was added to each tube. One hundred microliters of water was added directly into the other nine tubes set aside for the protease (−) to adjust the volumes so that all tubes contained combination volumes of approximately 750 μL. Thus, the combinations in the first set of tubes comprised a protease, a cationic detergent, a first chaotrope, and a second chaotrope; while the combinations in the second set of tubes comprised a first chaotrope, but no cationic detergent, protease, or second chaotrope. Both sets of combinations were transferred to an ABI PRISM 6100 Nucleic Acid PrepStation containing a GF/B glass fiber filter tray and vacuum filtered at 2.4 psi. It was observed that the combinations not comprising protease tended to clog the filters.

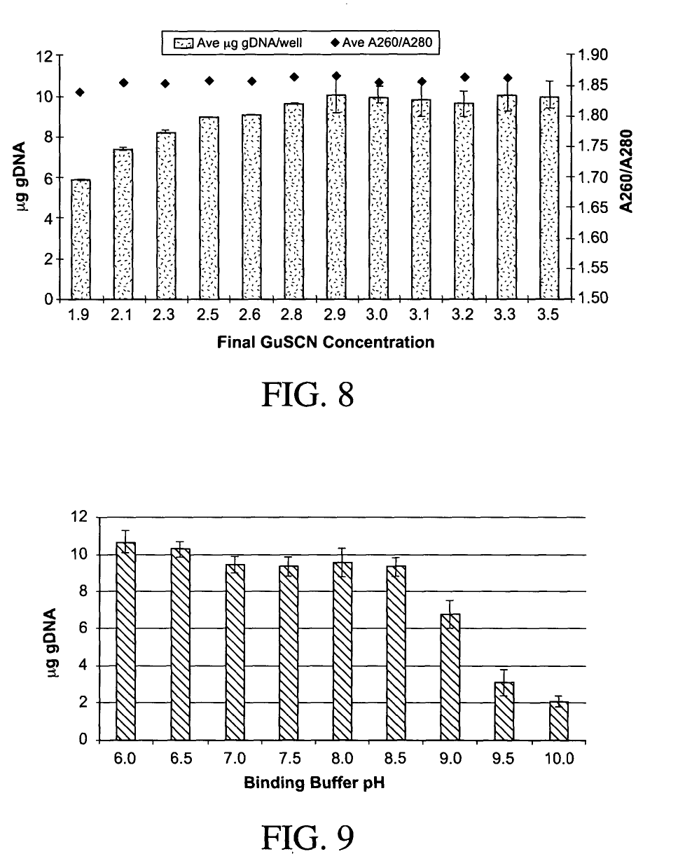

The wells were washed once with the chaotrope solution, then three times with 90% ethyl alcohol. The nucleic acid was eluted from the filter-bound Complexes using 100 μL 0.01 N NaOH solution, followed by 100 μL 15 mM Tris, 1 mM EDTA, pH 7.0. The eluate, containing the isolated nucleic acid, was collected in a 96-well archive tray. The eluted material was analyzed to evaluate the yield and purity (as determined from the A260/A280 ratio) of the isolated nucleic acid using a UV/V is Spectrophotometer. The results were plotted graphically as shown in FIG. 7.

### EXAMPLE 9

### Effect of Chaotrope Concentration on Nucleic Acid Isolation

To evaluate the effect of chaotrope concentration on the purity and yield of the isolated nucleic acid, increasing concentrations of GuSCN were tested with fresh whole blood samples. Two hundred microliter aliquots of fresh whole blood sample were placed into duplicate sets of twelve 1.5 mL microfuge tubes and 200 μL protease solution (2 mg proteinase K, 50 mM Tris-HCl pH 8.0 and 5 mM EDTA pH 8.0) was added. The tubes were incubated at 55° C. for 15 minutes, then varying volumes (250, 300, 350, 400, 450, 500, 550, 600, 650, 700, 750, 900 μL) of chaotrope solution (5M GuSCN, 20 mM EDTA pH 8.0, 60 mM Tris-HCl, pH 7.0 and 2% Zwittergent 3-16) were added. The final GuSCN concentrations in the combinations were 1.9, 2.1, 2.3, 2.5, 2.6, 2.8, 2.9, 3.0, 3.1, 3.2, 3.3, and 3.5 mM, respectively.

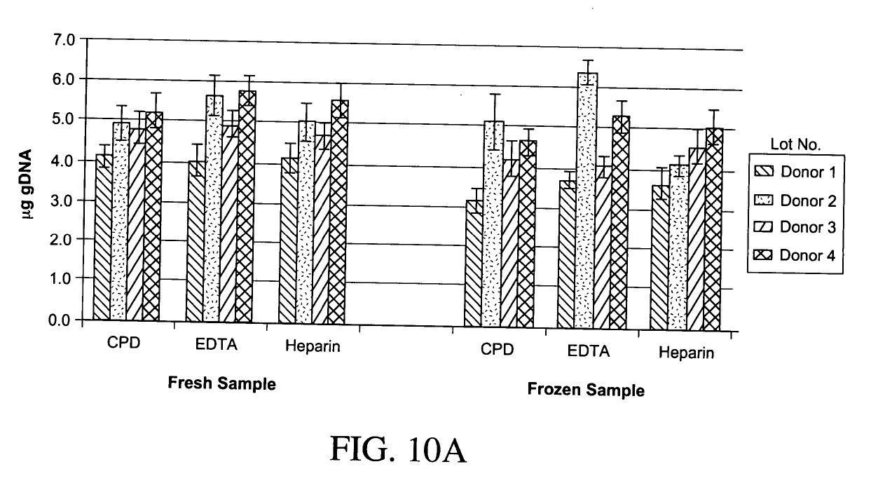

These combinations were transferred to individual wells of a GF/B glass fiber tray mounted on the ABI PRISM 6100 Nucleic Acid PrepStation and vacuum-filtered at 2.4 psi. The filters were washed once with 650 μL of the chaotrope solution, then three times with 90% ethyl alcohol. Nucleic acids were eluted from filter-bound Complexes using 100 μL 0.01 N NaOH solution, followed by 100 μL 15 mM Tris-HCl, 1 mM EDTA, pH 7.0. The eluate, containing the isolated nucleic acid, was collected in a 96-well sample archive tray. The eluate was analyzed by UV/V is spectrometer, as before, to determine the effect of varying concentrations of chaotrope on the yield and purity of the isolated nucleic acid. The results were plotted graphically, as shown in FIG. 8.

### EXAMPLE 10

### Evaluation of pH on Nucleic Acid Isolation

To evaluate whether the pH of the chaotrope solution buffer had an effect on the yield of isolated nucleic acid, several solution buffers were evaluated with fresh whole blood samples. Two hundred microliters of fresh whole blood sample was placed into three sets of nine 1.5 mL microfuge tubes and 200 μL of protease solution (2 mg proteinase K in 50 mM Tris-HCl pH 8.0 and 5 mM EDTA pH 8.0) was added to each tube. The tubes were incubated at 55° C. for 15 minutes, then 500 μL of one of nine different chaotrope solution was added, each chaotrope solution comprising 20 mM EDTA pH 8.0, 2% Zwittergent 3-16, 5M GuSCN and 60 mM of either: MES, pH 6.0 (Sigma M-2933); ACES, pH 6.5 (Calbiochem 104801); Tris-HCl, pH 7.0 (Sigma T-2413); Tris-HCl, pH 7.5 (Sigma T-2413); Tris-HCl, pH 8.0 (Sigma T-3088); Tris-HCl, pH 8.5 (Sigma T-2413); CHES, pH 9.0 (Sigma C-8210); CHES, pH 9.5 (Sigma C-8210); or AMP, pH 10.0 (Sigma 221).

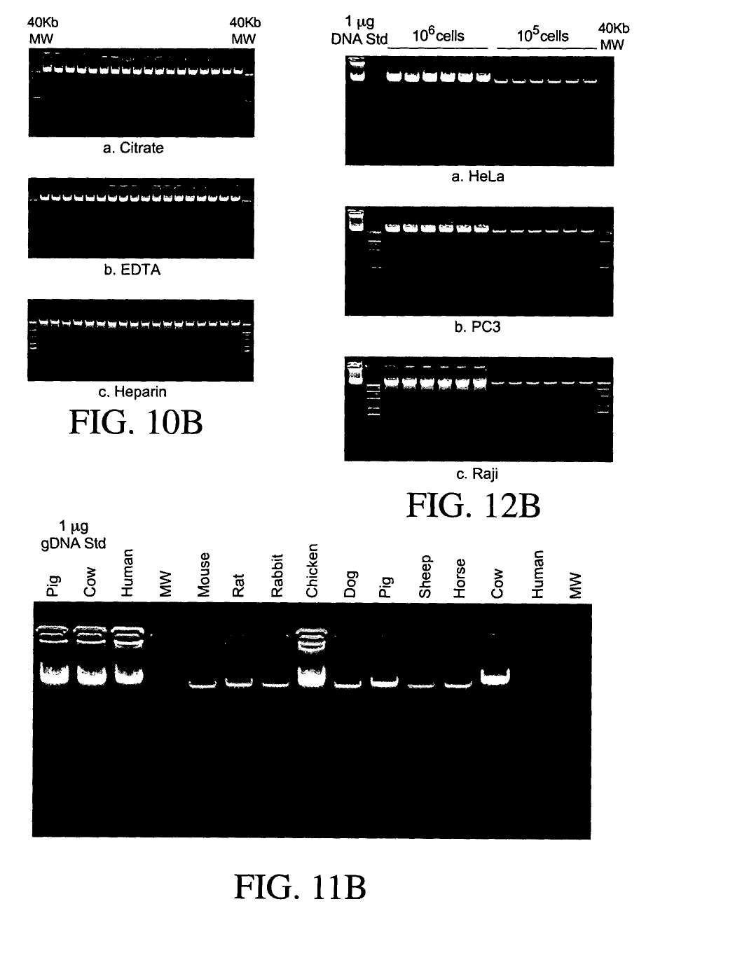

These combinations were transferred to individual wells of a GF/B glass fiber tray mounted on the ABI PRISM 6100 Nucleic Acid PrepStation and vacuum-filtered at 2.4 psi. The filters were washed once with the chaotrope solution containing the appropriate buffer, then three times with 90% ethyl alcohol. Nucleic acid was eluted from the filter-bound Complexes using 100 μL 0.01 N NaOH solution, followed by 100 μL 15 mM Tris-HCl, 1 mM EDTA, pH 7.0. The eluate, containing the isolated nucleic acid, was collected in a 96-well sample archive tray. The eluate was analyzed by UV/V is spectrometer, as before, and the results were plotted graphically, as shown in FIG. 9.

### EXAMPLE 11

### Effect of Anticoagulants on the Nucleic Acid Isolation from Blood Samples

One unit of whole blood was collected from each of four different donors. The blood from each donor was split into three approximately equal portions and a different anticoagulant (either citrate phosphate dextrose (CPD), EDTA or heparin) was added to each portion from each donor (“anticoagulated blood”). All of the anticoagulated blood samples were further split into two lots. One lot was stored at 4° C. (“fresh whole blood” in this exemplary embodiment) and the other lot was stored at −80° C. (“frozen whole blood” in this exemplary embodiment). The frozen whole blood samples were thawed and warmed to room temperature prior to use.

One hundred fifty microliter aliquots of either fresh whole blood sample or frozen whole blood sample, comprising one of the three different anticoagulants from one of the four donors were added to 32 separate wells in a 96-well deep well tray (Applied Biosystems P/N 4308641) and 100 μL of protease solution (300 ng proteinase K, 125 mM Tris-HCl, pH 8.0, 250 mM GuHCl and 0.4% CTAB) was added to each well. The tray was incubated, after mixing, in a 58° C. water bath for 10 minutes, then 500 μL of a chaotrope solution (5 M GuSCN, 50 mM EDTA pH 8.0, 200 mM Tris-HCl pH 7.0, 0.01% Antifoam A and 3% Zwittergent 3-16) was added and the combinations were mixed by pipetting each combination up and down 5 times using a multi-channel pipette (Matrix Cat. No. 2034). The combinations were transferred into individual wells of 96-well GF/B trays installed on the ABI PRISM 6100 Nucleic Acid PrepStation and vacuum filtered at 2.4 psi. The wells were washed once with 650 μL chaotrope solution, then three times with 90% ethyl alcohol. The nucleic acid was eluted from the filter-bound Complexes using 100 μL of 0.01 N NaOH solution, followed by 100 μL of 15 mM Tris-HCl, 1 mM EDTA, pH 7.0. The eluates from each well, containing the isolated nucleic acid, were collected in a 96-well archive tray. The quantities of nucleic acid recovered in the eluates were measured using a UV/V is Spectrophotometer (Hewlett Packard, Model 8453).

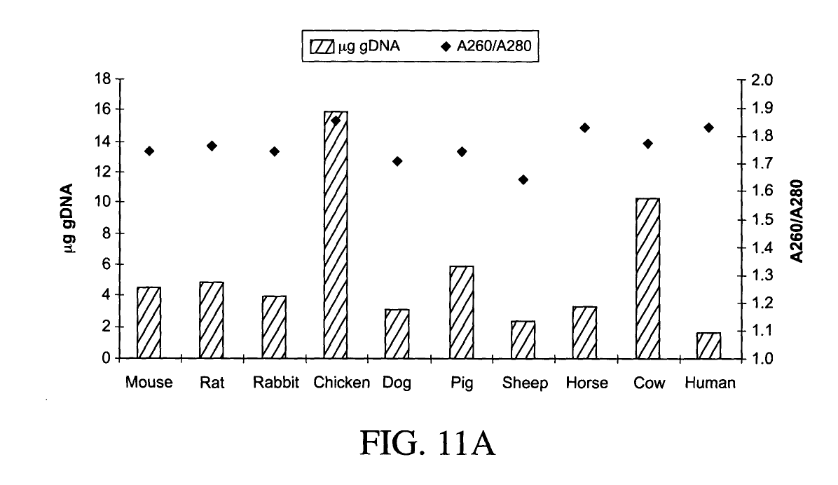

The yield of gDNA isolated from the samples is shown in FIG. 10. Based from the actual leukocyte counts of the samples, the results show 80-100% recovery of the expected gDNA content of the samples (data not shown). As shown in FIG. 10A, no significant differences were observed between the yield of nucleic acid isolated from the fresh and frozen blood samples or between the different anticoagulants. To evaluate the quality of the isolated gDNA, aliquots were electrophoresed on 1% agarose gels and the gels stained with ethidium bromide, as shown in FIG. 10B.

### EXAMPLE 12

### Isolation of Nucleic Acid from Animal Blood Samples

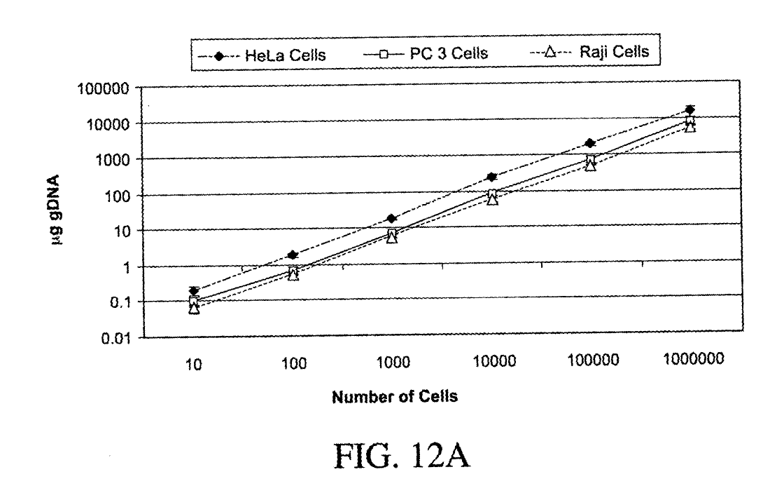

To verify that nucleic acid could be isolated from blood samples from other animal species using the methods of the invention, whole blood samples were obtained from sheep (ovine), rat, mouse, rabbit, pig, horse, dog, cow (bovine), and chicken. A human blood sample was also included as a control. A fifty microliter aliquot of one of these whole blood samples was placed into one of a series of microfuge tubes, mixed with 100 μL protease solution (300 ng proteinase K, 125 mM Tris-HCl, pH 8.0, 250 mM GuHCl and 0.4% CTAB), then incubated in a 58° C. water bath for 10 minutes. The tubes comprising the ovine and bovine samples were incubated an additional 20 minutes to complete cell lysis. Following incubation, 500 μL of a second chaotrope solution (5 M GuSCN, 50 mM EDTA pH 8.0, 200 mM Tris-HCl pH 7.0, 0.01% Antifoam A and 3% Zwittergent 3-16) was added and the combinations mixed by pipetting up and down 5 times. The combinations were transferred to individual wells of 96-well GF/B trays installed on the ABI PRISM 6100 Nucleic Acid PrepStation, and vacuum filtered at 2.4 psi. The wells were washed once with 650 μL of the chaotrope solution, then three times with 90% ethyl alcohol. The nucleic acid was eluted from the filter-bound Complexes using 100 μL 0.01 N NaOH solution, followed by 100 μL 15 mM Tris-HCl, 1 mM EDTA, pH 7.0. The eluates from each well, containing the isolated nucleic acid, were collected in a 96-well sample archive tray. The quantity of isolated nucleic acid was determined using a UV/V is Spectrophotometer (Hewlett Packard, Model 8453), see FIG. 11A. Twenty microliter aliquots of the isolated nucleic acids were analyzed by gel electrophoresis, as shown in FIG. 11B.

### EXAMPLE 13

### Isolation of Nucleic Acid from Tissue Culture Cells

Two human epithelial cell lines, HeLa cells (ATCC CCL-2) and PC-3 cells (ATCC CLR-1435), were grown as confluent monolayers in 75-cm² tissue culture flasks according to the methods and in the culture media recommended by ATCC. The cells were trypsinized following conventional protocols for adherent cell lines. The cells were centrifuged at 3000 rpm in an Eppendorf Model 5416 bench-top centrifuge and the resulting cell pellet was re-suspended in chilled phosphate-buffered saline (PBS; Sigma # P8357) to a final concentration of 1×10⁷ cells/mL. Human Raji cells (ATCC CCL-86) were grown in suspension culture according to the method and in the culture medium recommended by ATCC. The cells were centrifuged at 3000 rpm in an Eppendorf Model 5416 bench-top centrifuge and the resulting cell pellet was re-suspended in chilled phosphate-buffered saline (PBS; Sigma # P8357) to a final concentration of 1×10⁷ cells/mL.

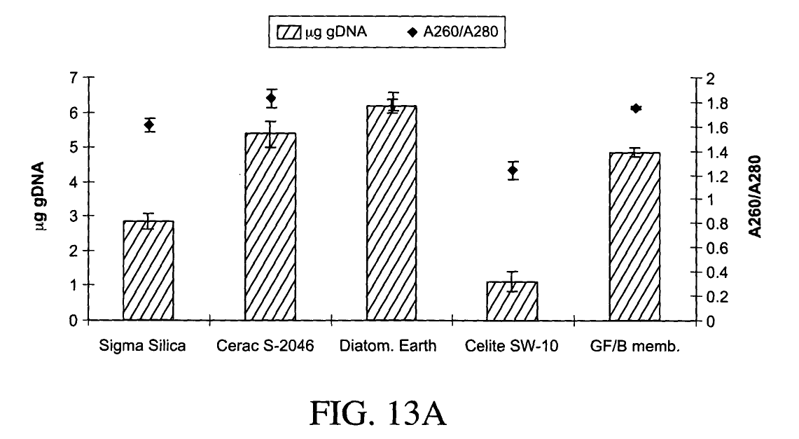

Aliquots of each of the three cell suspensions were diluted separately in chilled PBS to make a ten-fold dilution series ranging from 1×10⁷ cells/mL (undiluted) to 10 cells/mL, resulting in a total of 7 different cell dilutions (1×10⁷, 1×10⁶, 1×10⁵, 1×10⁴, 1000, 100, and 10 cells/mL). Three replicates of 100 μL aliquots of each dilution for each cell suspension was placed into individual wells of a deep well tray (Applied Biosystems P/N 4308641). Into each of the wells was added 400 μL of a chaotrope solution (5 M GuSCN, 50 mM EDTA pH 8.0, 200 mM Tris-HCl pH 7.0, 0.01% Antifoam A, and 3% Zwittergent 3-16) with mixing, to form combinations. The combinations were then transferred into individual wells of the 96-well GF/B filter tray installed on the ABI PRISM 6100 Nucleic Acid PrepStation and evacuated under 2.4 psi vacuum. The filters, comprising the bound Complexes, were washed once with the chaotrope solution, then three times with 90% ethanol. A one hundred microliter aliquot of 0.01N NaOH was added to each of the washed wells and incubated for three minutes at ambient temperature. The eluate was evacuated, collected into a 96-well sample archive tray, and neutralized by adding 100 μL 15 mM Tris-HCl, 1 mM EDTA pH 7.0 to the GF/B filter tray wells. The second eluates were collected into appropriate wells of the archive tray. The quantity of nucleic acid isolated from each cell sample was determined using a UV/V is Spectrophotometer (Hewlett Packard, Model #8453), see FIG. 12A. Aliquots of the isolated nucleic acid were also evaluated using gel electrophoresis, as shown in FIG. 12B.

### EXAMPLE 14

### Evaluation of Various Solid Phase Materials

Twenty milligrams of either silica particles from Sigma-Aldrich (Cat # S-5631), silica particles from CERAC (P/N S-2046), diatomaceous earth from J. T. Baker (P/N 1939-01; J. T. Baker, Philippsburg, N.J.) or Celite Grade SW-10 (Celite Corporation, Lompoc, Calif.) were placed into 1.5 mL microcentrifuge tubes, in triplicate.

Eighteen different microfuge tubes containing 150 μL frozen whole blood sample collected with citrate anticoagulant were each combined with 100 μL of protease solution (300 ng proteinase K (Applied Biosystems P/N 4333793), 125 mM Tris-HCl, pH 8.0 (Sigma), 255 mM GuHCl (Sigma) and 0.4% CTAB (Sigma)). These combinations were incubated at 58° C. for 10 minutes, then 500 μL of chaotrope solution (5 M GuSCN, 50 mM EDTA pH 8.0, 200 mM Tris-HCl, pH 7.0, 0.01% Antifoam A and 3% Zwittergent 3-16) was added with mixing. These combinations were transferred to the tubes containing the solid phase materials, described above, and mixed by vortexing. The tubes were incubated for 5 minutes at room temperature with constant shaking on the Mini Vortexer (VWR Scientific), allowing nucleic acid in the combination to bind to the solid phase. The tubes were then centrifuged for 5 minutes at 14,000 rpm in and Eppendorf Model 5804R benchtop centrifuge, the supernatant solutions were removed and the pellets were washed 2-3 times with 1 mL chaotrope solution, until the supernatant was clear.

The solid phase materials comprising the bound Complexes were washed twice with 1 mL 90% ethyl alcohol, then the solid phase materials were dried under vacuum in a Savant Speed Vac Plus, Model SC110A. The nucleic acid was eluted from the solid phase by adding 100 μL of 0.01 N NaOH into the tubes and constantly vortexing for 5 minutes, then 100 μL of 15 mM Tris-HCl (pH 7.0) was added to each tube. The tubes were then centrifuged for 5 minutes at 14,000 rpm in Eppendorf Model 5804R benchtop centrifuge and the supernatant solutions containing the nucleic acid were transferred to fresh microfuge tubes. As a control, aliquots of the same frozen whole blood sample were combined with the same protease solution and chaotrope solution, but with GF/B glass fiber tray solid phase was performed using filtration, as previously described.

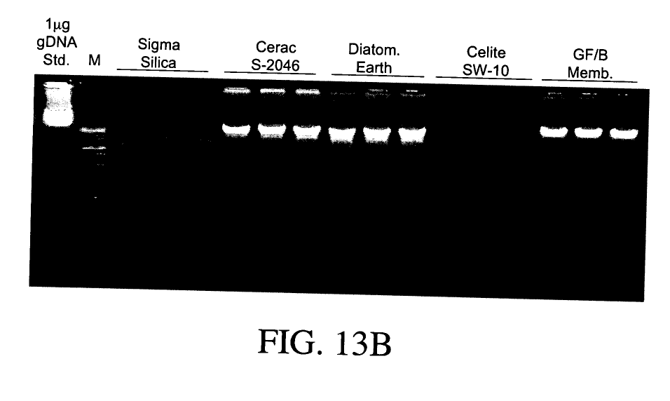

The yields and purity of the isolated nucleic acids were determined using a UV/V is Spectrophotometer, as before, and the results graphically displayed, as seen in FIG. 13A. The quality of the isolated nucleic acids was also evaluated by gel electrophoresis, as shown in FIG. 13B.

Although the invention has been described with reference to various applications, methods, and kits, it will be appreciated that various changes and modifications may be made without departing from the invention. The foregoing examples are provided to better illustrate the invention and are not intended to limit the scope of the invention.

1. A method for obtaining nucleic acid from a biological sample and binding the nucleic acid to a solid phase, comprising:
combining the sample with at least one protease and at least one zwitterionic compound to form a combination; and
exposing the combination to at least one solid phase to bind the nucleic acid.

2. The method of claim 1, further comprising at least one first chaotrope.

3. The method of claim 2, further comprising isolating the nucleic acid.

4. The method of claim 1, wherein the at least one zwitterionic compound comprises at least one of: n-Octyl-N,N-dimethyl-3-ammonio-1-propanesulfonate; n-Decyl-N,N-dimethyl-3-ammonio-1-propanesulfonate; n-Dodecyl-N,N-dimethyl-3-ammonio-1-propanesulfonate; n-Tetradecyl-N,N-dimethyl-3-ammonio-1-propanesulfonate; n-Hexadecyl-N,N-dimethyl-3-ammonio-1-propanesulfonate; n-Octadecyl-N,N-dimethyl-3-ammonio-1-propanesulfonate; Lauryldimethylamine N-oxide (LDAO™); 3-[(3-Cholamidopropyl)dimethylammonio]-1-propanesulfonate (CHAPS™); 3-[(3-Cholamidopropyl)dimethylammonio]-2-hydroxy-1-propanesulfonate (CHAPSO™); N,N-bis(3-D-Gluconamidopropyl)cholamide (BigCHAP™); N-Dodecyl-N,N-dimethylglycine (Empigen BB™); N-dodecyl-N,N-(dimethylammonio) butyrate (DDMAB™); ASB-14™; ASB-16™; dodecylbetaine; lauraminopropyl betaine; dipalmitoylphosphatidylcholine (lecithin); N-dodecyl-N,N-(dimethylammonio)undecanoate (DDMAU); n-decyl-N,N-dimethylglycine; N-octylphosphocholine; N-nonylphosphocholine; N-decylphosphocholine; N-dodecylphosphocholine; N-tetradecylphosphocholine; N-hexadecylphosphocholine; Octyl phospho-n-methylethanolamine; Decyl phospho-n-methylethanolamine; Dodecyl phospho-n-methylethanolamine; Cyclohexylethylphosphocholine; Cyclohexylpropylphosphocholine; Cyclohexylbutylphosphocholine, dimethylethylammonium propane sulfonate; 3-(1-pyridino)-1-propane sulfonate; dimethyl-(2-hydroxyethyl)-(3-sulfopropyl)-ammonium; 3-(1-methylpiperidinium)-1-sulfonate; dimethylbenzylammonium propane sulfonate; dimethylethyl-(3-sulfopropyl)-ammonium; or N-phenyl-methyl-N,N-dimethylammonium-propane-sulfonate.

5. The method of claim 3, further comprising at least one second chaotrope.

6. The method of claim 5, wherein the at least one first chaotrope, the at least one second chaotrope, or both the at least one first chaotrope and the at least one second chaotrope comprises at least one of: guanidine hydrochloride (GuHCl), guanidinium thiocyanate (GuSCN), urea, sodium bromide (NaBr), sodium iodide (NaI), sodium perchlorate (NaClO₄), sodium thiocyanate (NaSCN), potassium thiocyanate (KSCN), lithium chloride (LiCl), lithium bromide (LiBr), lithium iodide (LiI), potassium iodide (KI), potassium chloride (KCl), potassium bromide (KBr), tetrabutyl ammonium bromide, tetrabutyl ammonium chloride, tetrabutyl ammonium iodide, tetrapropyl ammonium bromide, tetrapropyl ammonium chloride, tetrapropyl ammonium iodide, or thiourea (NH₂CSNH₂).

7. The method of claim 3, further comprising at least one non-ionic detergent.

8. The method of claim 3, wherein the isolated nucleic acid comprises genomic deoxyribonucleic acid (gDNA).

9. The method of claim 2, further comprising at least one cationic detergent.

10. The method of claim 9, wherein the at least one cationic detergent comprises at least one of: cetyltrimethylammonium bromide (CTAB); cetyltrimethylammonium chloride (CTACl); tetradecyltrimethylammonium bromide (TTAB); tetradecyltrimethylammonium chloride (TTACl); dodecyltrimethylammonium bromide (DTAB); dodecyltrimethylammonium chloride (DTACl); dodecylethyldimethylammonium bromide (DEDTAB); decyltrimethylammonium bromide (D₁₀TAB); or dodecyltriphenylphosphonium bromide (DTPB).

11. The method of claim 2, further comprising at least one cationic detergent and at least one second chaotrope.

12. The method of claim 11, wherein the at least one cationic detergent comprises CTAB and the at least one second chaotrope comprises guanidium thiocyanate (GuSCN).

13. The method of claim 12, wherein the at least one protease comprises proteinase K; the at least one first chaotrope comprises guanine hydrochloride (GuHCl); the at least one zwitterionic compound comprises at least one of: n-Octyl-N,N-dimethyl-3-ammonio-1-propanesulfonate, n-Decyl-N,N-dimethyl-3-ammonio-1-propanesulfonate, n-Dodecyl-N,N-dimethyl-3-ammonio-1-propanesulfonate, n-Tetradecyl-N,N-dimethyl-3-ammonio-1-propanesulfonate, n-Hexadecyl-N,N-dimethyl-3-ammonio-1-propanesulfonate, n-Octadecyl-N,N-dimethyl-3-ammonio-1-propanesulfonate, Lauryldimethylamine N-oxide, N-Dodecyl-N,N-dimethylglycine, or N-dodecyl-N,N-(dimethylammonio)butyrate; and the sample comprises whole blood, white blood cells, buffy coat, or lysates, extracts, or material obtained therefrom.

14. A method for obtaining nucleic acid from a biological sample and binding the nucleic acid to a solid phase, comprising:
combining the sample with at least one chaotrope and at least one zwitterionic compound to form a combination; and
exposing the combination to at least one solid phase bind the nucleic acid.

15. The method of claim 14, further comprising isolating the nucleic acid.

16. The method of claim 15, wherein the at least one zwitterionic compound comprises at least one of: n-Octyl-N,N-dimethyl-3-ammonio-1-propanesulfonate; n-Decyl-N,N-dimethyl-3-ammonio-1-propanesulfonate; n-Dodecyl-N,N-dimethyl-3-ammonio-1-propanesulfonate; n-Tetradecyl-N,N-dimethyl-3-ammonio-1-propanesulfonate; n-Hexadecyl-N,N-dimethyl-3-ammonio-1-propanesulfonate; n-Octadecyl-N,N-dimethyl-3-ammonio-1-propanesulfonate; Lauryldimethylamine N-oxide (LDAO™); 3-[(3-Cholamidopropyl)dimethylammonio]-1-propanesulfonate (CHAPS™); 3-[(3-Cholamidopropyl)dimethylammonio]-2-hydroxy-1-propanesulfonate (CHAPSO™); N,N-bis(3-D-Gluconamidopropyl)cholamide (BigCHAP™); N-Dodecyl-N,N-dimethylglycine (Empigen BB™); N-dodecyl-N,N-(dimethylammonio)butyrate (DDMAB TM); ASB-14™; ASB-16™; dodecylbetaine; lauraminopropyl betaine; dipalmitoylphosphatidylcholine (lecithin); N-dodecyl-N,N-(dimethylammonio)undecanoate (DDMAU); n-decyl-N,N-dimethylglycine; N-octylphosphocholine; N-nonylphosphocholine; N-decylphosphocholine; N-dodecylphosphocholine; N-tetradecylphosphocholine; N-hexadecylphosphocholine; Octyl phospho-n-methylethanolamine; Decyl phospho-n-methylethanolamine; Dodecyl phospho-n-methylethanolamine; Cyclohexylethylphosphocholine; Cyclohexylpropylphosphocholine; Cyclohexylbutylphosphocholine, dimethylethylammonium propane sulfonate; 3-(1-pyridino)-1-propane sulfonate; dimethyl-(2-hydroxyethyl)-(3-sulfopropyl)-ammonium; 3-(1-methylpiperidinium)-1-sulfonate; dimethylbenzylammonium propane sulfonate; dimethylethyl-(3-sulfopropyl)-ammonium; or N-phenyl-methyl-N,N-dimethylammonium-propane-sulfonate.

17. The method of claim 15, wherein the at least one first chaotrope comprises at least one of: GuHCl; GuSCN; urea; NaBr; NaI; NaClO₄; NaSCN; LiCl; LiBr; LiI; KI; KCl; KBr; tetrabutyl ammonium bromide; tetrabutyl ammonium chloride; tetrabutyl ammonium iodide; tetrapropyl ammonium bromide; tetrapropyl ammonium chloride; tetrapropyl ammonium iodide; or NH₂CSNH₂.

18. The method of claim 15, further comprising at least one non-ionic detergent.

19. The method of claim 15, wherein the isolated nucleic acid comprises gDNA.

20. The method of claim 15, wherein the sample comprises at least one of: swabs, urine, sputum, saliva, semen, lymphatic fluid, amniotic fluid, cerebrospinal fluid, peritoneal effusions, fluid from cysts, synovial fluids, vitreous humor, aqueous humor, bursal fluid, eye washes, eye aspirates, plasma, serum, pulmonary lavage, lung aspirates, tissues, tissue culture cells, or lysates, extracts, or material obtained therefrom; the at least one chaotrope comprises GuSCN; and the at least one zwitterionic compound comprises at least one of: n-Octyl-N,N-dimethyl-3-ammonio-1-propanesulfonate, n-Decyl-N,N-dimethyl-3-ammonio-1-propanesulfonate, n-Dodecyl-N,N-dimethyl-3-ammonio-1-propanesulfonate, n-Tetradecyl-N,N-dimethyl-3-ammonio-1-propanesulfonate, n-Hexadecyl-N,N-dimethyl-3-ammonio-1-propanesulfonate, n-Octadecyl-N,N-dimethyl-3-ammonio-1-propanesulfonate, Lauryldimethylamine N-oxide, N-Dodecyl-N,N-dimethylglycine, or N-dodecyl-N,N-(dimethylammonio)butyrate.

21. A kit comprising, at least one zwitterionic compound; at least one chaotrope; and at least one solid phase.

22. The kit of claim 21, further comprising at least one protease.

23. The kit of claim 22, wherein the at least one protease comprises at least one of: Proteinase K, Pronase, subtilisin Carlsberg (subtilopeptidase A), thermolysin, pancreatic protease, Proteinase R, Protease T, Subtilisin A, Subtilisin DY, alkaline serine proteases from Streptomyces griseus or Bacillus licheniformis, dispase, thermitase, trypsin, chymotrypsin, Protease S, Protease Type XVIII from Rhizopus sp, Protease Type XIX, or Protease Type I.

24. The kit of claim 21, further comprising at least one cationic detergent.

25. The kit of claim 21, further comprising at least one wash solution.

26. The kit of claim 25, wherein the at least one wash solution comprises at least one alcohol.

27. The kit of claim 21, further comprising at least one elution solution.

28. The kit of claim 27, wherein the at least one elution solution comprises at least one alkaline solution, at least one alkaline buffer, or at least one alkaline solution and at least one alkaline buffer.

29. The kit of claim 28, wherein the at least one alkaline elution solution comprises at least one buffer, at least one sodium hydroxide (NaOH) solution, or at least one potassium hydroxide (KOH) solution.

30. The kit of claim 21, further comprising at least one buffer solution, at least one buffer salt, or at least one buffer solution and at least one buffer salt.

31. The kit of claim 21, wherein the at least one zwitterionic compound comprises at least one of: n-Octyl-N,N-dimethyl-3-ammonio-1-propanesulfonate; n-Decyl-N,N-dimethyl-3-ammonio-1-propanesulfonate; n-Dodecyl-N,N-dimethyl-3-ammonio-1-propanesulfonate; n-Tetradecyl-N,N-dimethyl-3-ammonio-1-propanesulfonate; n-Hexadecyl-N,N-dimethyl-3-ammonio-1-propanesulfonate; n-Octadecyl-N,N-dimethyl-3-ammonio-1-propanesulfonate; Lauryldimethylamine N-oxide (LDAO™); 3-[(3-Cholamidopropyl)dimethylammonio]-1-propanesulfonate (CHAPS™); 3-[(3-Cholamidopropyl)dimethylammonio]-2-hydroxy-1-propanesulfonate (CHAPSO™); N,N-bis(3-D-Gluconamidopropyl)cholamide (BigCHAP); N-Dodecyl-N,N-dimethylglycine (Empigen BB™); N-dodecyl-N,N-(dimethylammonio)butyrate (DDMAB™); ASB-14™; ASB-16™; dodecylbetaine; lauraminopropyl betaine; dipalmitoylphosphatidylcholine (lecithin); N-dodecyl-N,N-(dimethylammonio) undecanoate (DDMAU); n-decyl-N,N-dimethylglycine; N-octylphosphocholine; N-nonylphosphocholine; N-decylphosphocholine; N-dodecylphosphocholine; N-tetradecylphosphocholine; N-hexadecylphosphocholine; Octyl phospho-n-methylethanolamine; Decyl phospho-n-methylethanolamine; Dodecyl phospho-n-methylethanolamine; Cyclohexylethylphosphocholine; Cyclohexylpropylphosphocholine; Cyclohexylbutylphosphocholine, dimethylethylammonium propane sulfonate; 3-(1-pyridino)-1-propane sulfonate; dimethyl-(2-hydroxyethyl)-(3-sulfopropyl)-ammonium; 3-(1-methylpiperidinium)-1-sulfonate; dimethylbenzylammonium propane sulfonate; dimethylethyl-(3-sulfopropyl)-ammonium; or N-phenyl-methyl-N,N-dimethylammonium-propane-sulfonate; the at least one chaotrope comprises at least one of: GuHCl; GuSCN; urea; NaBr; NaI; NaClO₄; NaSCN; LiCl; LiBr; LiI; KI; KCl; KBr; tetrabutyl ammonium bromide; tetrabutyl ammonium chloride; tetrabutyl ammonium iodide; tetrapropyl ammonium bromide; tetrapropyl ammonium chloride; tetrapropyl ammonium iodide; or NH₂CSNH₂; and the at least one solid phase comprises at least one of: silica particles; silicon dioxide; silica gel; fumed silica; diatomaceous earth; glass; alkylsilica; aluminum silicate; sodium silicate, zirconium silicate; quartz; or sand.
